(19) United States
(12) Patent Application Publication
(10) Pub. No.: US 2011/0112142 A1
(43) Pub. Date: May 12, 2011

(54) NOVEL ESTROGEN RECEPTOR LIGANDS

(76) Inventors: Daniel Nöteberg, Vasterhaninge (SE); Elisabet Kallin, Tullinge (SE); Mattias Wennerstål, Hagersten (SE)

(21) Appl. No.: 12/736,416

(22) PCT Filed: Apr. 8, 2009
(86) PCT No.: PCT/EP2009/054220

§ 371 Date: Jan. 24, 2011

(30) Foreign Application Priority Data
Apr. 11, 2008 (GB) 0806656.5

(51) Int. Cl. A61K 31/4709; C07D 307/80; C07D 307/81; C07D 409/04; C07D 409/14; C07D 407/04; C07D 405/04; C07D 413/04; C07D 417/04; A61K 31/343; A61K 31/381; A61K 31/416; A61K 31/443; A61K 31/422; A61K 31/427; A61P 19/10; A61P 19/08; A61P 3/00; A61P 9/00; A61P 25/00; A61P 3/04; A61P 13/00; A61P 25/22; A61P 25/24; A61P 29/00; A61P 15/00; A61P 9/12; A61P 35/00; A61P 27/02

(52) U.S. Cl. 514/314; 549/469; 549/60; 548/361.1; 546/284.1; 546/167; 549/58; 548/247; 548/203; 514/469; 514/444; 514/406; 514/337; 514/443; 514/378; 514/365

(57) ABSTRACT

The invention provides a pharmaceutical composition comprising a compound of formula (I) or a pharmaceutically acceptable ester, amide, solvate or salt thereof, including a salt of such an ester or amide, and a solvate of such an ester, amide or salt: wherein R³ is selected from the group consisting of OR^{A}; —CHO; —C(O)C₁₋₄alkyl; —C(O)phenyl; —O—C(O)R^{A}; and N(R^{B})2; R⁶ is selected from certain cyclic groups defined in the specification; and the remaining groups are defined in the specification; together with a pharmaceutically acceptable carrier. Most of the compounds are novel. The invention also provides the use of such compounds in the treatment or prophylaxis of a condition associated with a disease or disorder associated with estrogen receptor activity.

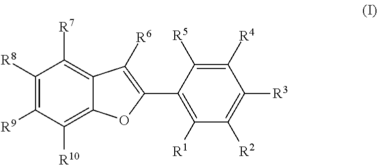

### FIELD OF INVENTION

This invention relates to compounds which are estrogen receptor ligands and are preferably selective for the estrogen receptor β isoform, to methods of preparing such compounds and to methods for using such compounds in treatment of diseases related to the estrogen receptor such as depressive disorders, anxiety disorders, Alzheimer's disease, cognitive disorders, osteoporosis, elevated blood triglyceride levels, atherosclerosis, endometriosis, urinary incontinence, autoimmune disease, and cancer of the lung, colon, breast, uterus and prostate.

### BACKGROUND OF INVENTION

The estrogen receptor (ER) is a ligand activated mammalian transcription factor involved in the up and down regulation of gene expression. The natural hormone for the estrogen receptor is β-17-estradiol (E2) and closely related metabolites. Binding of estradiol to the estrogen receptor causes a dimerization of the receptor and the dimer in turn binds to estrogen response elements (ERE's) on DNA. The ER/DNA complex recruits other transcription factors responsible for the transcription of DNA downstream from the ERE into mRNA which is eventually translated into protein. Alternatively the interaction of ER with DNA may be indirect through the intermediacy of other transcription factors, most notably fos and jun. Since the expression of a large number of genes is regulated by the estrogen receptor and since the estrogen receptor is expressed in many cell types, modulation of the estrogen receptor through binding of either natural hormones or synthetic ER ligands can have profound effects on the physiology and pathophysiology of the organism.

Historically it has been believed there was only one estrogen receptor. However a second subtype (ER-β) has been discovered. While both the “classical” ER-α and the more recently discovered ER-β are widely distributed in different tissues, they nevertheless display markedly different cell type and tissue distributions. Therefore synthetic ligands which are either ER-α or ER-β selective may preserve the beneficial effects of estrogen while reducing the risk of undesirable side effects.

Estrogens are critical for sexual development in females. In addition, estrogens play an important role in maintaining bone density, regulation of blood lipid levels, and appear to have neuroprotective effects. Consequently decreased estrogen production in post-menopausal women is associated with a number of diseases such as osteoporosis, atherosclerosis, depression and cognitive disorders. Conversely certain types of proliferative diseases such as breast and uterine cancer and endometriosis are stimulated by estrogens and therefore antiestrogens (i.e., estrogen antagonists) have utility in the prevention and treatment of these types of disorders.

The efficacy of the natural estrogen, 17β-estradiol, for the treatment of various forms of depressive illness has also been demonstrated and it has been suggested that the anti-depressant activity of estrogen may be mediated via regulation of tryptophan hydroxylase activity and subsequent serotonin synthesis (See, e.g., Lu N Z, Shlaes T A, Cundlah C, Dziennis S E, Lyle R E, Bethea C L, “Ovarian steroid action on tryptophan hydroxylase protein and serotonin compared to localization of ovarian steroid receptors in midbrain of guinea pigs.” Endocrine 11:257-267, 1999). The pleiotropic nature of natural estrogen preclude its widespread, more chronic use due to the increased risk of proliferative effects on breast, uterine and ovarian tissues. The identification of the estrogen receptor, ERβ, has provided a means by which to identify more selective estrogen agents which have the desired anti-depressant activity in the absence of the proliferative effects which are mediated by ERα. Thus, it has been shown that therapeutic agents having ERβ-selectivity are potentially particularly effective in the treatment of depression.

Certain benzofuran compounds are known per se from European Journal of Organic Chemistry, 2005, (12), 2481-2490; Australian Journal of Chemistry 1999, 52(8), 767-774; Bulletin de la Societe Chimique de France, 1974, (9-10 pt. 2), 2225-2232; and Comptes Rendus des Seances de l'Academie des Sciences, Serie C: Sciences Chimiques, 1967, 265(5), 320-323. None of these documents disclose that the compounds have any biological activity.

Certain benzofurans are known to have biological activity at estrogen receptors, see Anti-cancer Drug Design 1991, 6(5), 417-426; DE 41 17 512; WO 03/051860; and WO 2007/047204.

What is needed in the art are compounds that can produce the same positive responses as estrogen replacement therapy without the negative side effects. Also needed are estrogen-like compounds that exert selective effects on different tissues of the body.

The compounds used in the present invention are ligands for estrogen receptors and as such may be useful for treatment or prevention of a variety of conditions related to estrogen functioning including bone loss, bone fractures, osteoporosis, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression, autoimmune disease, and lung, colon, breast, uterus, and prostate cancer.

### SUMMARY OF THE INVENTION

The present inventors have found that certain benzofuran compounds have valuable biological activity. Those compounds comprise a compound of formula (I) or a pharmaceutically acceptable ester, amide, solvate or salt thereof, including a salt of such an ester or amide, and a solvate of such an ester, amide or salt:

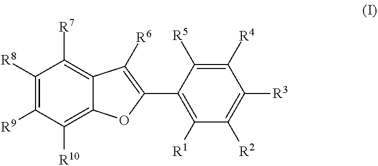

wherein R¹, R², R⁴ and R⁵ are each independently selected from the group consisting of hydrogen, OR^{A}, halogen, cyano, nitro, C₁₋₆alkyl, C₂₋₆alkenyl, C₂₋₆alkynyl, haloC₁₋₆alkyl, dihaloC₁₋₆alkyl and trihaloC₁₋₆alkyl;
R³ is selected from the group consisting of OR^{A}; —CHO; —C(O)C₁₋₄alkyl; —C(O)phenyl; —O—C(O)R^{A}; and N(R^{B})₂ in which each R^{B} is independently selected from the group consisting of hydrogen, —C(O)C₁₋₄alkyl, —C(O)phenyl, —SO₂C₁₋₄alkyl, —SO₂phenyl, C₁₋₆alkyl, C₂₋₆alkenyl, C₂₋₆alkynyl, C₃₋₈cycloalkyl, C₃₋₈cycloalkyl-C₁₋₆alkyl, C₆₋₁₀aryl, C₆₋₁₀aryl-C₁₋₆alkyl, C₅₋₁₀heterocyclyl and C₅₋₁₀heterocyclyl-C₁₋₆ alkyl;
or R³ and R⁴ together with the atoms to which they are attached, form a 5-, 6- or 7-membered cyclic group optionally containing one to three heteroatoms selected from O, N and S, said 5-, 6- or 7-membered cyclic group being optionally substituted with 1 or 2 groups selected from OR^{A}, halogen, cyano, nitro, C₁₋₆alkyl, C₂₋₆alkenyl, C₂₋₆alkynyl, haloC₁₋₆alkyl, dihaloC₁₋₆alkyl and trihaloC₁₋₆alkyl;
R⁶ is selected from the group consisting of C₃₋₈cycloalkyl, C₃₋₈cycloalkyl-C₁₋₆alkyl, C₃₋₈cycloalkyl-C₁₋₆alkenyl, phenyl, biphenyl, phenyl-C(═CH₂)—, and C₅₋₁₀heterocyclyl, wherein said phenyl, biphenyl, phenyl-C(═CH₂)— or C₅₋₁₀heterocyclyl group is unsubstituted or substituted on the ring with 1 to 3 substituents, each substituent being selected from the group consisting of OR^{A}; halogen; cyano; nitro; —CHO; —C(O)C₁₋₆alkyl; C₁₋₆alkyl, C₁₋₆alkoxy or C₁₋₆alkoxyalkyl optionally substituted by 1 to 3 halogen atoms; C₂₋₆alkenyl optionally substituted by halogen or cyano; C₂₋₆alkynyl; SO₂H; SO₂C₁₋₆alkyl; SH; and SC₁₋₆alkyl;

### R⁸ is OR^{A};

R⁷, R⁹ and R¹⁰ are each independently selected from the group consisting of hydrogen, OR^{A}, halogen, cyano, nitro, C₁₋₆alkyl, C₂₋₆alkenyl, C₂₋₆alkynyl, C(O)H, C(O)C₁₋₆alkyl, haloC₁₋₆alkyl, dihaloC₁₋₆alkyl, trihaloC cyanoC₁₋₆alkyl, and C₁₋₄alkoxyC₁₋₆alkyl; and
R^{A} is selected from the group consisting of hydrogen, C₁₋₆alkyl, C₂₋₆alkenyl, C₂₋₆alkynyl, C₃₋₈cycloalkyl, C₃₋₈cycloalkyl-C₁₋₆alkyl, C₆₋₁₀aryl and C₆₋₁₀ aryl-C₁₋₆alkyl.

The compounds defined above have been found to be ligands of the estrogen receptor. The compounds accordingly have use in the treatment or prophylaxis of conditions associated with estrogen receptor activity.

Accordingly the invention provides a pharmaceutical composition which comprises a compound as defined above together with a pharmaceutically acceptable carrier; a compound as defined above for use as a medicament; use of a compound as defined above for the manufacture of a medicament for the treatment or prophylaxis of a condition associated with a disease or disorder associated with estrogen receptor activity; and a method for the treatment or prophylaxis of a disease or disorder associated with estrogen receptor activity in a mammal, which comprises administering to the mammal a therapeutically effective amount of a compound as defined above. Most of the compounds as defined above are novel, and the invention therefore further provides those compounds per se, said compounds being those defined above with the provisos that (i) if all of R², R³, R⁸ and R⁹ represent methoxy groups and all of R¹, R⁴, R⁵, R⁷ and R¹⁰ represent hydrogen atoms, then R⁶ represents a group other than phenyl or 3,4-dimethoxyphenyl; (ii) if both R³ and R⁸ represent methoxy groups, R² represents an isopropoxy group, R⁹ represents a hydroxy or an isopropoxy group, and all of R¹, R⁴, R⁵, R⁷ and R¹⁰ represent hydrogen atoms, then R⁶ represents a group other than 3,4,5-trimethoxyphenyl; and (iii) if both of R³ and R⁸ represent methoxy groups and all of R¹, R², R⁴, R⁵, R⁷, R⁹ and R¹⁰ represent hydrogen atoms, then R⁶ represents a group other than phenyl.

### DETAILED DESCRIPTION OF INVENTION

The compounds used in the invention may contain chiral (asymmetric) centers or the molecule as a whole may be chiral. The individual stereoisomers (enantiomers and diastereoisomers) and mixtures of these are within the scope of the present invention.

The present invention uses compounds that are estrogen receptor ligands. The term “estrogen receptor ligand” as used herein is intended to cover any moiety which binds to an estrogen receptor. The ligand may act as an agonist, a partial agonist, an antagonist or a partial antagonist. The ligand may be ERβ selective or display mixed ERα and ERβ activity. For example, the ligand may act both as an agonist or a partial agonist of ERβ and as an antagonist or a partial antagonist of ERα. In general, however, the compounds have an improved combination of activity (generally agonist activity, most of the compounds being) and selectivity for the β receptor.

Preferably R^{A} is selected from the group consisting of hydrogen, C₁₋₄alkyl, C₃₋₆cycloalkyl, C₃₋₆cycloalkyl-C₁₋₄ alkyl, phenyl and benzyl. More preferably, R^{A} is selected from the group consisting of hydrogen, C₁₋₄alkyl and C₃₋₆cycloalkyl. Most preferably, R^{A} is selected from the group consisting of hydrogen and C₁₋₄alkyl, for example R^{A} may be hydrogen.

Preferably R¹, R², and R⁵ are each independently selected from the group consisting of hydrogen, OR^{A}, halogen, cyano, nitro, C₁₋₄alkyl, haloC₁₋₄alkyl, dihaloC₁₋₄alkyl and trihaloC₁₋₄alkyl. More preferably R¹, R² and R⁵ are each independently selected from the group consisting of hydrogen, OR^{A}, halogen, cyano, halomethyl, dihalomethyl and trihalomethyl. Most preferably R¹, R², and R⁵ are each independently selected from hydrogen, hydroxy, halogen (for example chlorine or fluorine), cyano, methyl and trifluoromethyl. Preferably at least 1, for example at least 2, for example all 3, of R¹, R², and R⁵ represent hydrogen.

In one preferred embodiment, R³ is selected from the group consisting of OR^{A} and N(R^{B})₂, in which R^{A} has one of the preferred meanings given above and each R^{B} is independently selected from the group consisting of hydrogen, C₁₋₄alkyl, and C₃₋₆cycloalkyl, especially hydrogen and C₁₋₄alkyl. Especially, R³ represents a hydroxy group. In one embodiment, when R³ represents —OC₁₋₄alkyl, suitably R⁸ represents a group other than —OC₁₋₄alkyl; for example when R³ represents —OCH₃, suitably R⁸ represents a group other than —OCH₃.

In one preferred embodiment, R⁴ represents one of the preferred groups mentioned above for R¹, R², and R⁵.

In a further preferred embodiment, R³ and R⁴, together with the atoms to which they are attached, form a 5-, 6- or 7-membered cyclic group optionally containing one to three heteroatoms selected from O, N and S, preferably N, said 5-, 6- or 7-membered cyclic group being optionally substituted with 1 or 2 groups selected from OR^{A}, halogen, cyano, nitro, C₁₋₆alkyl, C₂₋₆alkenyl, C₂₋₆alkynyl, haloC₁₋₆alkyl, dihaloC₁₋₆alkyl and trihaloC₁₋₆alkyl. Preferably R³ and R⁴ together represent an —NH—CH═N—, —NH—N═CH— or —CH—CH—NH— group, in which case said heterocycle together with the phenyl ring to which it is fused is a benzimidazole, indole or, especially, indazole, group. In a heterocycle comprising R³ and R⁴ together, the atom adjacent the phenyl ring in the R³ position is preferably a heteroatom, especially a nitrogen atom. A heterocycle comprising R³ and R⁴ is preferably unsubstituted.

R⁶ is preferably selected from the group consisting of C₃₋₆cycloalkyl, C₃₋₆cycloalkyl-C₁₋₂alkyl, C₃₋₆cycloalkyl-C₁₋₂alkenyl, phenyl, biphenyl, phenyl-C(═CH₂)—, and C₅₋₇heterocyclyl wherein said phenyl, biphenyl, phenyl-C(═CH₂)— or C₅₋₁₀heterocyclyl group is unsubstituted or substituted on the ring with 1 or 2 substituents, each substituent being selected from the group consisting of OR^{A}; halogen (for example chlorine or fluorine); cyano; and C₁₋₄alkyl or C₁₋₄alkoxy optionally substituted by 1 to 3 halogen atoms. Most preferably, R⁶ is an aromatic group, for example a phenyl or aromatic C₅heterocyclyl group, with optional substitution of said group as described above. Preferred C₅heterocyclyl groups include thiophenyl, thiazolyl, furanyl, pyrazolyl, pyrrolyl, oxazolyl and imidazolyl, especially furanyl, thiophenyl and pyrazolyl. For example, R⁶ may represent a phenyl group or a C₅heterocyclyl group optionally substituted by one of the above substituents.

R⁷, R⁹ and R¹⁰ are preferably each independently selected from the group consisting of hydrogen, OR^{A}, halogen, cyano, C₁₋₄alkyl, haloC₁₋₄ alkyl, dihaloC₁₋₄alkyl and trihaloC₁₋₄ alkyl. More preferably, R⁷, R⁹ and R¹⁰ are each independently selected from the group consisting of hydrogen, OR^{A}, halogen, cyano, halomethyl, dihalomethyl and trihalomethyl. Most preferably R⁷, R⁹ and R¹⁰ are each independently selected from hydrogen, hydroxy, halogen (for example chlorine or fluorine), cyano, methyl and trifluoromethyl. Preferably at least 1, for example at least 2, for example all 3, of R⁷, R⁹ and R¹⁰ represent hydrogen.

Accordingly, in one preferred group of compounds:

R^{A} represents C₁₋₄alkyl or, especially, hydrogen;
R¹, R², and R⁵ are each independently selected from the group consisting of hydrogen, OR^{A}, halogen (for example chlorine or fluorine), cyano, halomethyl, dihalomethyl and trihalomethyl; preferably at least 1, for example at least 2, for example all 3, of R¹, R², and R⁵ represent hydrogen;
R³ represents N(R^{B})₂, in which each R^{B} independently represents hydrogen and C₁₋₄alkyl, or, preferably, OR^{A};
R⁴ represents one of the preferred groups mentioned above for R¹, R², and R⁵; or
R³ and R⁴ together represent an —NH—CH═N—, —CH═CH—NH— or, especially, —NH—N═CH— group;
R⁶ represents an aromatic group, for example a phenyl or a C₅heterocyclyl group, which can either be unsubstituted or substituted on the ring with 1 or 2 substituents, each substituent being selected from the group consisting of OR^{A}; halogen (for example chlorine or fluorine); cyano; and C₁₋₄alkyl or C₁₋₄alkoxy optionally substituted by 1 to 3 halogen atoms;
R⁷, R⁹ and R¹⁰ each independently represent hydrogen, OR^{A}, halogen (for example chlorine or fluorine), cyano, halomethyl, dihalomethyl and trihalomethyl, especially hydrogen, hydroxy, halogen, cyano, methyl and trifluoromethyl, preferably at least 1, for example at least 2, for example all 3, of R⁷, R⁹ and R¹⁰ being hydrogen.

Compounds of the formula I include, but are not limited to, the following:
2-(4-Hydroxy-phenyl)-7-methyl-3-phenyl-benzofuran-5-ol;
5-Hydroxy-2-(4-hydroxy-phenyl)-3-phenyl-benzofuran-7-carbonitrile;
2-(2-Fluoro-4-hydroxy-phenyl)-7-methyl-3-phenyl-benzofuran-5-ol;
7-Dibromomethyl-2-(4-hydroxy-phenyl)-3-phenyl-benzofuran-5-ol;
[5-Hydroxy-2-(4-hydroxy-phenyl)-3-phenyl-benzofuran-7-yl]-acetonitrile;
2-(4-Hydroxy-phenyl)-7-(1-methoxy-ethyl)-3-phenyl-benzofuran-5-ol;
7-Difluoromethyl-2-(4-hydroxy-phenyl)-3-phenyl-benzofuran-5-ol;
2-(4-Hydroxy-phenyl)-3-phenyl-7-vinyl-benzofuran-5-ol;
2-(4-Hydroxy-phenyl)-3-thiophen-3-yl-7-trifluoromethyl-benzofuran-5-ol;
7-Fluoro-2-(1H-indazol-5-yl)-3-thiophen-3-yl-benzofuran-5-ol;
2-[7-Chloro-5-hydroxy-2-(4-hydroxy-phenyl)-benzofuran-3-yl]-furan-3-carbonitrile;
2-(4-Hydroxy-phenyl)-7-methyl-3-thiophen-3-yl-benzofuran-5-ol;
3-(2,5-Difluoro-phenyl)-2-(4-hydroxy-phenyl)-7-methyl-benzofuran-5-ol;
3-[5-Hydroxy-2-(4-hydroxy-phenyl)-7-methyl-benzofuran-3-yl]-benzonitrile;
2-(4-Hydroxy-phenyl)-7-methyl-3-m-tolyl-benzofuran-5-ol;
2-(4-Hydroxy-phenyl)-7-methyl-3-thiophen-2-yl-benzofuran-5-ol;
2-(4-Hydroxy-phenyl)-7-methyl-3-pyridin-3-yl-benzofuran-5-ol;
2-[5-Hydroxy-2-(4-hydroxy-phenyl)-7-methyl-benzofuran-3-yl]-benzonitrile;
2-(4-Hydroxy-phenyl)-7-methyl-3-(3-nitro-phenyl)-benzofuran-5-ol;
5-[5-Hydroxy-2-(4-hydroxy-phenyl)-7-methyl-benzofuran-3-yl]-thiophene-2-carbaldehyde;
3-(3,5-Difluoro-phenyl)-2-(4-hydroxy-phenyl)-7-methyl-benzofuran-5-ol;
3-(3,5-Dichloro-phenyl)-2-(4-hydroxy-phenyl)-7-methyl-benzofuran-5-ol;
2-(4-Hydroxy-phenyl)-7-methyl-3-(2-phenoxy-phenyl)-benzofuran-5-ol;
3-Biphenyl-2-yl-2-(4-hydroxy-phenyl)-7-methyl-benzofuran-5-ol;
3-(2-Hydroxy-phenyl)-2-(4-hydroxy-phenyl)-7-methyl-benzofuran-5-ol;
1-{3-[5-Hydroxy-2-(4-hydroxy-phenyl)-7-methyl-benzofuran-3-yl]-phenyl}-ethanone;
2-(4-Hydroxy-phenyl)-3-(3-methanesulfonyl-phenyl)-7-methyl-benzofuran-5-ol;
3-(3-Ethylsulfanyl-phenyl)-2-(4-hydroxy-phenyl)-7-methyl-benzofuran-5-ol;
2-(4-Hydroxy-phenyl)-7-methyl-3-quinolin-5-yl-benzofuran-5-ol;
1-{5-[5-Hydroxy-2-(4-hydroxy-phenyl)-7-methyl-benzofuran-3-yl]-thiophen-2-yl}-ethanone;
2-(4-Hydroxy-phenyl)-7-methyl-2′,3′-dihydro-[3,5]bibenzofuranyl-5-ol;
2-(4-Hydroxy-phenyl)-7-methyl-3-(3-trifluoromethoxy-phenyl)-benzofuran-5-ol;
3-(2-Fluoro-pyridin-3-yl)-2-(4-hydroxy-phenyl)-7-methyl-benzofuran-5-ol;
3-Benzo[b]thiophen-2-yl-2-(4-hydroxy-phenyl)-7-methyl-benzofuran-5-ol;
2-(4-Hydroxy-phenyl)-7-methyl-3-(1-methyl-1H-pyrazol-4-yl)-benzofuran-5-ol;
2-(4-Hydroxy-phenyl)-7-methyl-3-(1-phenyl-vinyl)-benzofuran-5-ol;
2-(4-Hydroxy-phenyl)-7-methyl-3-pyridin-4-yl-benzofuran-5-ol;
2-(4-Hydroxy-phenyl)-7-methyl-3-(1-methyl-1H-pyrrol-2-yl)-benzofuran-5-ol;
3-(3,5-Dimethyl-isoxazol-4-yl)-2-(4-hydroxy-phenyl)-7-methyl-benzofuran-5-ol;
3-(5-Fluoro-2-methyl-phenyl)-2-(4-hydroxy-phenyl)-7-methyl-benzofuran-5-ol;
3-[1-(4-Fluoro-phenyl)-vinyl]-2-(4-hydroxy-phenyl)-7-methyl-benzofuran-5-ol;
3-Cyclopropyl-2-(4-hydroxy-phenyl)-7-methyl-benzofuran-5-ol;
3-(5-Fluoro-2-methoxy-phenyl)-2-(4-hydroxy-phenyl)-7-methyl-benzofuran-5-ol;
2-(4-Hydroxy-phenyl)-7-methyl-3-(1H-pyrrol-2-ye-benzofuran-5-ol;
3-Furan-2-yl-2-(4-hydroxy-phenyl)-7-methyl-benzofuran-5-ol;
2-(4-Hydroxy-phenyl)-7-methyl-3-thiazol-5-yl-benzofuran-5-ol;
2-(4-Hydroxy-phenyl)-3-(2-methoxy-thiazol-4-yl)-7-methyl-benzofuran-5-ol;
2-(4-Hydroxy-phenyl)-7-methyl-3-thiazol-2-yl-benzofuran-5-ol;
2-(4-Hydroxy-phenyl)-3-(2-isopropyl-phenyl)-7-methyl-benzofuran-5-ol;
3-(2-Ethyl-phenyl)-2-(4-hydroxy-phenyl)-7-methyl-benzofuran-5-ol;
(E)-3-{2-[5-Hydroxy-2-(4-hydroxy-phenyl)-7-methyl-benzofuran-3-yl]-phenyl}-acrylonitrile;
3-(2-Butoxy-phenyl)-2-(4-hydroxy-phenyl)-7-methyl-benzofuran-5-ol;
2-(4-Hydroxy-phenyl)-7-methyl-3-(2-trifluoromethoxy-phenyl)-benzofuran-5-ol;
4-[5-Hydroxy-2-(4-hydroxy-phenyl)-7-methyl-benzofuran-3-yl]-thiophene-2-carbaldehyde;
3-((E)-2-Cyclopropyl-vinyl)-2-(4-hydroxy-phenyl)-7-methyl-benzofuran-5-ol;
2-(4-Hydroxy-phenyl)-7-methyl-3-(3-methyl-thiophen-2-yl)-benzofuran-5-ol;
2-[5-Hydroxy-2-(4-hydroxy-phenyl)-7-methyl-benzofuran-3-yl]-thiophene-3-carbaldehyde;
2-[5-Hydroxy-2-(4-hydroxy-phenyl)-7-methyl-benzofuran-3-yl]-thiophene-3-carbonitrile;
7-Bromomethyl-2-(4-hydroxy-phenyl)-3-phenyl-benzofuran-5-ol;
5-Hydroxy-2-(4-hydroxy-phenyl)-3-thiophen-3-yl-benzofuran-7-carbonitrile;
5-Hydroxy-2-(4-hydroxy-phenyl)-3-phenyl-benzofuran-7-carbaldehyde;
7-Chloro-2-(4-hydroxy-phenyl)-3-phenyl-benzofuran-5-ol;
7-Chloro-2-(4-hydroxy-phenyl)-3-thiophen-3-yl-benzofuran-5-ol;
5-Hydroxy-2-(4-hydroxy-phenyl)-3-thiophen-3-yl-benzofuran-7-carbaldehyde;
2-(4-Hydroxy-phenyl)-3-thiophen-3-yl-7-vinyl-benzofuran-5-ol;
2-[7-Chloro-5-hydroxy-2-(4-hydroxy-phenyl)-benzofuran-3-yl]-thiophene-3-carbonitrile;
3-(3-Cyano-furan-2-yl)-5-hydroxy-2-(4-hydroxy-phenyl)-benzofuran-7-carbonitrile;
2-[5-Hydroxy-2-(4-hydroxy-phenyl)-7-trifluoromethyl-benzofuran-3-yl]-furan-3-carbonitrile;
2-[2-(3-Fluoro-4-hydroxy-phenyl)-5-hydroxy-7-methyl-benzofuran-3-yl]-furan-3-carbonitrile;
2-(4-Hydroxy-phenyl)-6-methyl-3-phenyl-benzofuran-5-ol;
2-(2,5-Difluoro-4-hydroxy-phenyl)-7-methyl-3-phenyl-benzofuran-5-ol;
2-(2,6-Difluoro-4-hydroxy-phenyl)-7-methyl-3-phenyl-benzofuran-5-ol;
2-(3-Fluoro-4-hydroxy-phenyl)-7-methyl-3-phenyl-benzofuran-5-ol;
2-(1H-Indazol-5-yl)-7-methyl-3-phenyl-benzofuran-5-ol;
2-(3,5-Difluoro-4-hydroxy-phenyl)-7-methyl-3-phenyl-benzofuran-5-ol;
2-(3-Chloro-5-fluoro-4-hydroxy-phenyl)-7-methyl-3-phenyl-benzofuran-5-ol;
5-(5-Methoxy-7-methyl-3-phenyl-benzofuran-2-yl)-1H-indazole;
2-(3-Fluoro-4-hydroxy-phenyl)-7-methyl-3-thiophen-3-yl-benzofuran-5-ol;
2-(2-Fluoro-4-hydroxy-phenyl)-7-methyl-3-thiophen-3-yl-benzofuran-5-ol;
2-(2,6-Difluoro-4-hydroxy-phenyl)-7-methyl-3-thiophen-3-yl-benzofuran-5-ol;
2-(3-Chloro-5-fluoro-4-hydroxy-phenyl)-7-methyl-3-thiophen-3-yl-benzofuran-5-ol;
2-[2-(3-Chloro-5-fluoro-4-hydroxy-phenyl)-5-hydroxy-7-methyl-benzofuran-3-yl]-furan-3-carbonitrile; and
2-[7-Fluoro-5-hydroxy-2-(1H-indazol-5-yl)-benzofuran-3-yl]-furan-3-carbonitrile.

The compound names given above were generated in accordance with IUPAC by the ACD Labs 8.0/name program, version 8.05 and/or with ISIS DRAW Autonom 2000.

Depending upon the substituents present in compounds of the formula I, the compounds may form esters, amides and/or salts. Salts and solvates of compounds of formula (I) which are suitable for use in medicine are those wherein a counterion or associated solvent is pharmaceutically acceptable. However, salts and solvates having non-pharmaceutically acceptable counterions or associated solvents are within the scope of the present invention, for example, for use as intermediates in the preparation of the compounds of formula (I) and their pharmaceutically acceptable salts, solvates and physiologically functional derivatives. By the term “physiologically functional derivative” is meant a chemical derivative of a compound of formula (I) having the same physiological function as the free compound of formula (I), for example, by being convertible in the body thereto. Esters and amides are examples of physiologically functional derivatives.

Suitable salts include those formed by reaction of basic groups in the compound of formula I with organic or inorganic acids. In particular, suitable salts include those formed with mineral acids, strong organic carboxylic acids, such as alkanecarboxylic acids of 1 to 4 carbon atoms which are unsubstituted or substituted, for example, by halogen, such as saturated or unsaturated dicarboxylic acids, such as hydroxycarboxylic acids, such as amino acids, or with organic sulfonic acids, such as (C₁-C₄)-alkyl- or aryl-sulfonic acids which are unsubstituted or substituted, for example by halogen. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycolic, lactic, salicylic, oxaloacetic, methanesulfonic, ethanesulfonic, p-toluenesulfonic, formic, benzoic, malonic, naphthalene-2-sulfonic, benzenesulfonic, isethionic, ascorbic, malic, phthalic, aspartic, and glutamic acids, lysine and arginine. Other acids such as oxalic, while not in themselves pharmaceutically acceptable, may be useful as intermediates in obtaining the compounds and their pharmaceutical acceptable acid addition salts.

Pharmaceutically acceptable esters and amides of the compounds of formula (I) may have an appropriate group, for example an OH group or an NR³ group, converted by reaction with an appropriate acid.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”.

A compound which, upon administration to the recipient, is capable of being converted into a compound of formula (I) as described above, or an active metabolite or residue thereof, is known as a “prodrug”. A prodrug may, for example, be converted within the body, e.g. by hydrolysis in the blood, into its active form that has medical effects. Pharmaceutical acceptable prodrugs are described in T. Higuchi and V. Stella, Prodrugs as Novel Delivery Systems, Vol. 14 of the A. C. S. Symposium Series (1976); “Design of Prodrugs” ed. H. Bundgaard, Elsevier, 1985; and in Edward B. Roche, ed., Bioreversible Carriers in Drug Design, American Pharmaceutical Association and Pergamon Press, 1987, which are incorporated herein by reference.

The following definitions apply to the terms as used throughout this specification, unless otherwise limited in specific instances.

As used herein, the term “alkyl” means both straight and branched chain saturated hydrocarbon groups. Examples of alkyl groups include methyl, ethyl, n-propyl, iso-propyl, n-butyl, t-butyl, i-butyl, sec-butyl, pentyl and hexyl groups. Among unbranched alkyl groups, there are preferred methyl, ethyl, n-propyl, iso-propyl, n-butyl groups. Among branched alkyl groups, there may be mentioned t-butyl, i-butyl, 1-ethylpropyl and 1-ethylbutyl groups.

As used herein, the term “alkoxy” means the group O-alkyl, where “alkyl” is used as described above. Examples of alkoxy groups include methoxy and ethoxy groups. Other examples include propoxy and butoxy.

As used herein, the term “alkenyl” means both straight and branched chain unsaturated hydrocarbon groups with at least one carbon carbon double bond. Examples of alkenyl groups include ethenyl, propenyl, butenyl, pentenyl and hexenyl. Preferred alkenyl groups include ethenyl, 1-propenyl and 2-propenyl.

As used herein, the term “alkynyl” means both straight and branched chain unsaturated hydrocarbon groups with at least one carbon carbon triple bond. Examples of alkynyl groups include ethynyl, propynyl, butynyl, pentynyl and hexynyl. Preferred alkynyl groups include ethynyl 1-propynyl and 2-propynyl.

As used herein, the term “cycloalkyl” means a saturated group in a ring system. A cycloalkyl group can be monocyclic or bicyclic. A bicyclic group may, for example, be fused or bridged. Examples of monocyclic cycloalkyl groups include cyclopropyl, cyclobutyl and cyclopentyl.

Other examples of monocyclic cycloalkyl groups are cyclohexyl, cycloheptyl and cyclooctyl. Examples of bicyclic cycloalkyl groups include bicyclo[2.2.1]hept-2-yl. Preferably, the cycloalkyl group is monocyclic.

As used herein, the term “aryl” means a monocyclic or bicyclic aromatic carbocyclic group. Examples of aryl groups include phenyl and naphthyl. A naphthyl group may be attached through the 1 or the 2 position. In a bicyclic aromatic group, one of the rings may, for example, be partially saturated. Examples of such groups include indanyl and tetrahydronaphthyl. Specifically, the term C₅₋₁₀ aryl is used herein to mean a group comprising from 5 to 10 carbon atoms in a monocyclic or bicyclic aromatic group. A particularly preferred C₅₋₁₀ aryl group is phenyl.

As used herein, the term “halogen” means fluorine, chlorine, bromine or iodine. Fluorine, chlorine and bromine are particularly preferred.

As used herein, the term “haloalkyl” means an alkyl group having a halogen substituent, the terms “alkyl” and “halogen” being understood to have the meanings outlined above. Similarly, the term “dihaloalkyl” means an alkyl group having two halogen substituents and the term “trihaloalkyl” means an alkyl group having three halogen substituents. Examples of haloalkyl groups include fluoromethyl, chloromethyl, bromomethyl, fluoromethyl, fluoropropyl and fluorobutyl groups; examples of dihaloalkyl groups include difluoromethyl and difluoroethyl groups; examples of triihaloalkyl groups include trifluoromethyl and trifluoroethyl groups.

As used herein, the term “heterocyclyl” means an aromatic or a non-aromatic cyclic group of carbon atoms wherein from one to three of the carbon atoms is/are replaced by one or more heteroatoms independently selected from nitrogen, oxygen or sulfur. A heterocyclyl group may, for example, be monocyclic or bicyclic. In a bicyclic heterocyclyl group there may be one or more heteroatoms in each ring, or only in one of the rings. A heteroatom is preferably O or N. Heterocyclyl groups containing a suitable nitrogen atom include the corresponding N-oxides. Examples of monocyclic heterocycloalkyl rings include aziridinyl, azetidinyl, pyrrolidinyl, imidazolidinyl, pyrazolidinyl, piperidinyl, piperazinyl, tetrahydrofuranyl, tetrahydropyranyl, morpholinyl, thiomorpholinyl and azepanyl.

Examples of bicyclic heterocyclic rings in which one of the rings is non-aromatic include dihydrobenzofuranyl, indanyl, indolinyl, isoindolinyl, tetrahydroisoquinolinyl, tetrahydroquinolyl and benzoazepanyl.

Examples of monocyclic heteroaryl groups include furanyl, thienyl, pyrrolyl, oxazolyl, thiazolyl, imidazolyl, oxadiazolyl, thiadiazolyl, pyridyl, triazolyl, triazinyl, pyridazyl, pyrimidinyl, isothiazolyl, isoxazolyl, pyrazinyl, pyrazolyl and pyrimidinyl; examples of bicyclic heteroaryl groups include quinoxalinyl, quinazolinyl, pyridopyrazinyl, benzoxazolyl, benzothiophenyl, benzimidazolyl, naphthyridinyl, quinolinyl, benzofuranyl, indolyl, benzothiazolyl, oxazolyl[4,5-b]pyridiyl, pyridopyrimidinyl, isoquinolinyl and benzodroxazole.

Examples of preferred heterocyclyl groups include piperidinyl, tetrahydrofuranyl, tetrahydropyranyl, pyridyl, pyrimidyl and indolyl. Preferred heterocyclyl groups also include thiophenyl, thiazolyl, furanyl, pyrazolyl, pyrrolyl and imidazolyl.

As used herein the term “cycloalkylalkyl” means a group cycloalkyl-alkyl- attached through the alkyl group, “cycloalkyl” and “alkyl” being understood to have the meanings outlined above.

As mentioned above, the compounds defined herein have activity as estrogen receptor ligands. The compounds have activity as estrogen receptor modulators, and may be agonists, partial agonists, antagonists, or partial antagonists of the estrogen receptor.

The compounds may thus be used in the treatment of diseases or disorders associated with estrogen receptor activity. In particular, the compounds that are agonists or partial agonists of the estrogen receptor (which includes most of the compounds of the invention) may be used in the treatment of diseases or disorders for which selective agonists or partial agonists of the estrogen receptor are indicated. Compounds that are antagonists or partial antagonists of the estrogen receptor may be used in the treatment of diseases or disorders for which selective antagonists or partial antagonists of the estrogen receptor are indicated.

Clinical conditions for which an agonist or partial agonist is indicated include, but are not limited to, bone loss, bone fractures, osteoporosis, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression, autoimmune disease, inflammation, IBD, IBS, sexual dysfunction, hypertension, retinal degeneration, and lung, colon, breast, uterus, and prostate cancer, and/or disorders related to estrogen functioning.

The compounds find particular application in the treatment or prophylaxis of the following: bone loss, bone fractures, osteoporosis, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression, autoimmune disease, inflammation IBD, IBS, sexual dysfunction, hypertension, retinal degeneration, and lung, colon, breast, uterus, and prostate cancer, and/or disorders related to estrogen functioning.

The amount of active ingredient which is required to achieve a therapeutic effect will, of course, vary with the particular compound, the route of administration, the subject under treatment, including the type, species, age, weight, sex, and medical condition of the subject and the renal and hepatic function of the subject, and the particular disorder or disease being treated, as well as its severity. An ordinarily skilled physician, veterinarian or clinician can readily determine and prescribe the effective amount of the drug required to prevent, counter or arrest the progress of the condition.

Oral dosages of the present invention, when used for the indicated effects, will range between about 0.01 mg per kg of body weight per day (mg/kg/day) to about 100 mg/kg/day, preferably 0.01 mg per kg of body weight per day (mg/kg/day) to 10 mg/kg/day, and most preferably 0.1 to 5.0 mg/kg/day, for adult humans. For oral administration, the compositions are preferably provided in the form of tablets or other forms of presentation provided in discrete units containing 0.01, 0.05, 0.1, 0.5, 1.0, 2.5, 5.0, 10.0, 15.0, 25.0, 50.0, 100, and 500 milligrams of the active ingredient for the symptomatic adjustment of the dosage to the patient to be treated. A medicament typically contains from about 0.01 mg to about 500 mg of the active ingredient, preferably from about 1 mg to about 100 mg of active ingredient. Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

While it is possible for the active ingredient to be administered alone, it is preferable for it to be present in a pharmaceutical formulation or composition. Accordingly, the invention provides a pharmaceutical formulation comprising a compound of the general formula I or a pharmaceutically acceptable ester, amide, solvate or salt thereof, including a salt of such an ester or amide, and a solvate of such asn ester amide or salt, and a pharmaceutically acceptable diluent, excipient or carrier (collectively referred to herein as “carrier” materials). Pharmaceutical compositions of the invention may take the form of a pharmaceutical formulation as described below.

The pharmaceutical formulations according to the invention include those suitable for oral, parenteral (including subcutaneous, intradermal, intramuscular, intravenous [bolus or infusion], and intraarticular), inhalation (including fine particle dusts or mists which may be generated by means of various types of metered does pressurized aerosols), nebulizers or insufflators, rectal, intraperitoneal and topical (including dermal, buccal, sublingual, and intraocular) administration, although the most suitable route may depend upon, for example, the condition and disorder of the recipient.

The formulations may conveniently be presented in unit dosage form and may be prepared by any of the methods well known in the art of pharmacy. All methods include the step of bringing the active ingredient into association with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets, pills or tablets each containing a predetermined amount of the active ingredient; as a powder or granules; as a solution or a suspension in an aqueous liquid or a non-aqueous liquid, for example as elixirs, tinctures, suspensions or syrups; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein. The present compounds can, for example, be administered in a form suitable for immediate release or extended release. Immediate release or extended release can be achieved by the use of suitable pharmaceutical compositions comprising the present compounds, or, particularly in the case of extended release, by the use of devices such as subcutaneous implants or osmotic pumps. The present compounds can also be administered liposomally.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate, calcium sulfate, sorbitol, glucose and/or lactose and/or other excipients, binders, extenders, disintegrants, diluents and lubricants such as those known in the art. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Disintegrators include without limitation starch, methylcellulose, agar, bentonite, xanthan gum and the like. The compounds of formula (I) can also be delivered through the oral cavity by sublingual and/or buccal administration. Molded tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use. Lubricants used in these dosage forms include sodium oleate, sodium stearate, magnesium stearate, sodium benzoate, sodium acetate, sodium chloride and the like. For oral administration in liquid form, the oral drug components can be combined with any oral, non-toxic, pharmaceutically acceptable inert carrier such as ethanol, glycerol, water, and the like.

The compounds can also be administered in the form of liposome delivery systems, such as small unilamellar vesicles, large unilamellar vesicles and multilamellar vesicles. Liposomes can be formed from a variety of phospholipids, 1,2-dipalmitoylphosphatidylcholine, phosphatidyl ethanolamine (cephaline), or phosphatidylcholine (lecithin).

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

Exemplary compositions for nasal, aerosol or inhalation administration include solutions in saline, which can contain, for example, benzyl alcohol or other suitable preservatives, absorption promoters to enhance bioavailability, and/or other solubilizing or dispersing agents such as those known in the art.

Formulations for rectal administration may be presented as a suppository with the usual carriers such as cocoa butter, synthetic glyceride esters or polyethylene glycol. Such carriers are typically solid at ordinary temperatures, but liquefy and/or dissolve in the rectal cavity to release the drug.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerine or sucrose and acacia. Exemplary compositions for topical administration include a topical carrier such as Plastibase (mineral oil gelled with polyethylene).

Preferred unit dosage formulations are those containing an effective dose, as hereinbefore recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavouring agents.

Whilst a compound as defined herein may be used as the sole active ingredient in a medicament, it is also possible for the compound to be used in combination with one or more further active agents. Such further active agents may be further compounds as defined herein, or they may be different therapeutic agents, for example an antidepressant, an anxiolytic, an anti-psychotic, or an agent useful in the prevention or treatment of osteoporosis or other pharmaceutically active material. For example, the compounds as defined herein may be effectively administered in combination with effective amounts of other agents such as an antidepressant, an anxiolytic, an anti-psychotic, an organic bisphosphonate or a cathepsin K inhibitor. Nonlimiting examples of antidepressants include noradrenaline reuptake inhibitors (NRI), selective serotonin reuptake inhibitors, monoamine oxidase inhibitors, tricyclic antidepressants (TCA), dopamine reuptake inhibitors (DRI), opioids, selective seretonic reuptake enhancers, tetracyclic antidepressants, reversible inhibitors of monoamine oxidase, melatonin agonists, serotonin and noradrenaline reuptake inhibitors (SNRI), corticotropin releasing factor antagonists, α-adrenoreceptor antagonists, 5HT1α receptor agonists and antagonists, lithium and atypical anti-psychotics. Examples of antidepressants of the SSRI class include Fluoxetine and Sertraline; examples of antidepressants of the SNRI class Venlafaxine, Citalopram, Paroxetine, Escitalopram, Fluvoxamine; examples of antidepressants of the SNRI class include Duloxetine; examples of antidepressants of the DRI and NRI classes include Bupropion; examples of antidepressants of the TCA class include Amitriptyline and Dothiepin (Dosulepin). Examples of atypical antipsychotics include: Clozapine, Olanzapine, Risperidone, Quetiapine, Ziprasidone and Dopamine partial agonists. Nonlimiting examples of anxiolytics include benzodiazepines and non-benzodiazapines. Examples of benzodiazapines include lorazepam, alprazolam, and diazepam. Examples of non-benzodiazapines include Buspirone (Buspar®), barbiturates and meprobamate. One or more of those further anti-depressants may be used in combination.

Nonlimiting examples of said organic bisphosphonates include adendronate, clodronate, etidronate, ibandronate, incadronate, minodronate, neridronate, risedronate, piridronate, pamidronate, tiludronate, zoledronate, pharmaceutically acceptable salts or esters thereof, and mixtures thereof. Preferred organic biphosphonates include alendronate and pharmaceutically acceptable salts and mixtures thereof. Most preferred is alendronate monosodium trihydrate.

The precise dosage of the bisphosphonate will vary with the dosing schedule, the oral potency of the particular bisphosphonate chosen, the age, size, sex and condition of the mammal or human, the nature and severity of the disorder to be treated, and other relevant medical and physical factors. Thus, a precise pharmaceutically effective amount cannot be specified in advance and can be readily determined by the caregiver or clinician. An appropriate amount can be determined by routine experimentation from animal models and human clinical studies. Generally, an appropriate amount of bisphosphonate is chosen to obtain a bone resorption inhibiting effect, i.e. a bone resorption inhibiting amount of the bisphonsphonate is administered. For humans, an effective oral dose of bisphosphonate is typically from about 1.5 to about 6000 μg/kg of body weight and preferably about 10 to about 2000 μg/kg of body weight.

For human oral compositions comprising alendronate, pharmaceutically acceptable salts thereof, or pharmaceutically acceptable derivatives thereof, a unit dosage typically comprises from about 8.75 mg to about 140 mg of the alendronate compound, on an alendronic acid active weight basis, i.e. on the basis of the corresponding acid.

The compounds as defined herein can be used in combination with other agents useful for treating estrogen-mediated conditions. The individual components of such combinations can be administered separately at different times during the course of therapy or concurrently in divided or single combination forms. The present invention is therefore to be understood as embracing all such regimes of simultaneous or alternating treatment and the term “administering” is to be interpreted accordingly. It will be understood that the scope of combinations of the compounds of this invention with other agents useful for treating estrogen-mediated conditions includes in principle any combination with any pharmaceutical composition useful for treating disorders related to estrogen functioning.

The above other therapeutic agents, when employed in combination with the compounds as defined herein, may be used, for example, in those amounts indicated in the Physicians' Desk Reference (PDR) or as otherwise determined by one of ordinary skill in the art.

Where the compounds are utilized in combination with one or more other therapeutic agent(s), either concurrently or sequentially, the following combination ratios and dosage ranges are preferred:

When combined with an antidepressant, an anxiolytic, an anti-psychotic, an organic bisphosphonate or a cathepsin K inhibitor, the compounds defined herein may be employed in a weight ratio to the additional agent within the range from about 10:1 to about 1:10.

The compounds of the invention as described above also find use, optionally in labelled form, as a diagnostic agent for the diagnosis of conditions associated with malfunction of the estrogen receptor. For example, such a compound may be radioactively labelled.

The compounds of the invention as described above, optionally in labelled form, also find use as a reference compound in methods of discovering other agonists, partial agonists, antagonists or partial antagonists of the estrogen receptor. Thus, the invention provides a method of discovering a ligand of the estrogen receptor which comprises use of a compound as defined herein such a compound of the invention in labelled form, as a reference compound. For example, such a method may involve a competitive binding experiment in which binding of a compound to the estrogen receptor is reduced by the presence of a further compound which has estrogen receptor-binding characteristics, for example stronger estrogen receptor-binding characteristics than the compound of the invention in question.

Numerous synthetic routes to the compounds of the present invention can be devised by any person skilled in the art and the possible synthetic routes described below do not limit the invention. Many methods exist in the literature for the synthesis of benzofurans, and a number of possible synthetic routes are shown schematically below. Where appropriate, any initially produced compound as defined herein can be converted into another compound as defined herein by known methods.

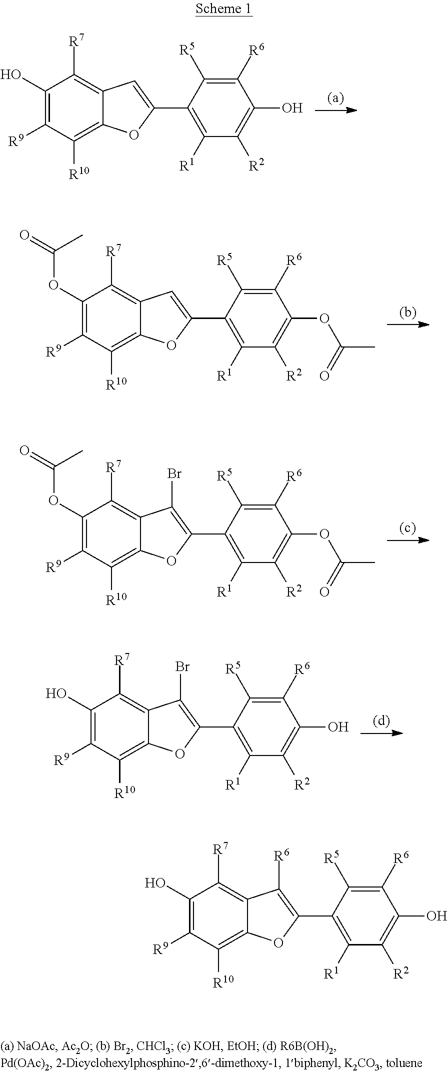

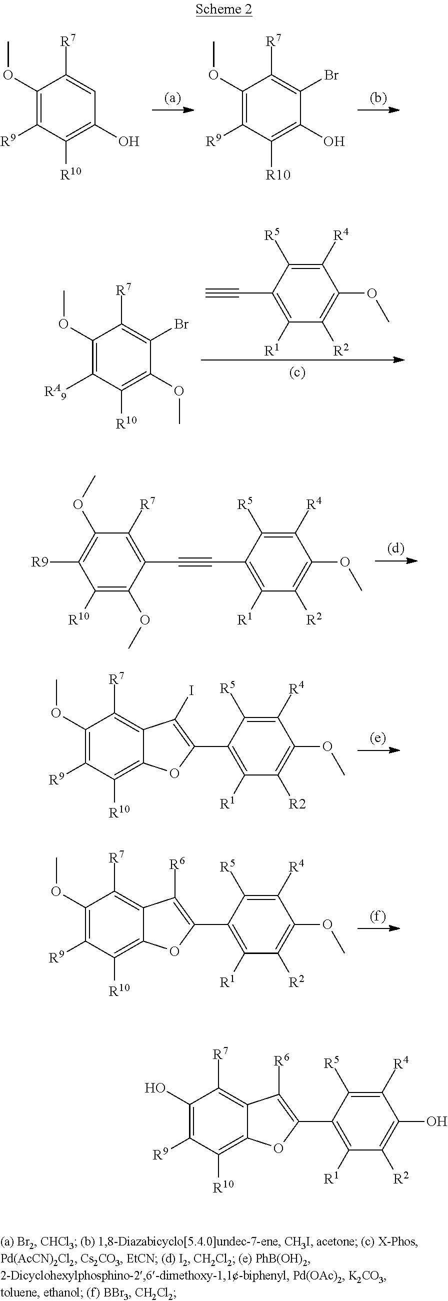

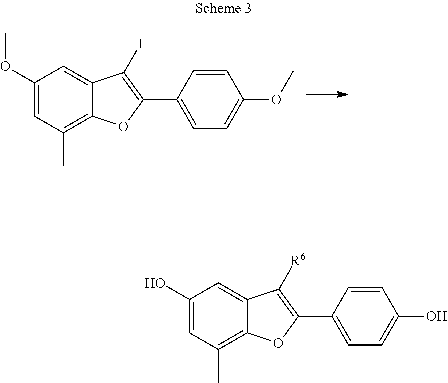

When using Scheme 3, the following general process was used. Boronic acids (0.1 mmol, 2 eq) were weighed into microwave vials. Sodium carbonate (11 mg, 0.1 mmol, 2 eq) was dissolved in water (0.5 ml) and added. The benzofuran (20 mg, 0.05 mmol, 1 eq) was dissolved in DME (1 ml) and added. Tetrakis (2 mg, 0.0015 mmol, 0.03 eq) was dissolved in ethanol (0.5 ml), and added. The vials were flushed with nitrogen, capped and irradiated in a microwave oven at 140 degrees for 3 minutes. The reaction mixtures were filtered into prep. HPLC vials, and chromatographed in acidic buffer using 20-100% water/acetonitrile gradient. UV-peaks in the range 16-24 minutes were collected. For the majority of the reactions a major uv-peak elutes at around 18 min. Fraction tubes were evaporated, the residues dissolved in acetone and transferred to preweighed vials. The vials were evaporated to dryness, kept under vacuum overnight and weighed.

The dried compounds were dissolved in DCM (1 ml) containing cyclohexene (21 ul) in vials with septa, flushed with nitrogen and cooled in alumina blocks in dry ice/acetone bath. Boron tribromide (1 M solution in DCM, 200 uL) was added with a syringe. The vials were allowed to reach 10 degrees and then kept at 4 degrees over night.

The reactions were cooled, quenched with MeOH (100 ul), diluted with DCM containing 10% MeOH (1 ml), and washed with sat.sodium bicarbonate. The sodium bicarbonate solution was washed once more with DCM (1 ml). The phases were separated using a phase separation membrane. The organic phase was evaporated to dryness and subjected to reversed phase preparative HPLC. Appropriate fractions were combined and evaporated, and identified by 1H-NMR and LC/MS. Purity was determined by analytical HPLC.

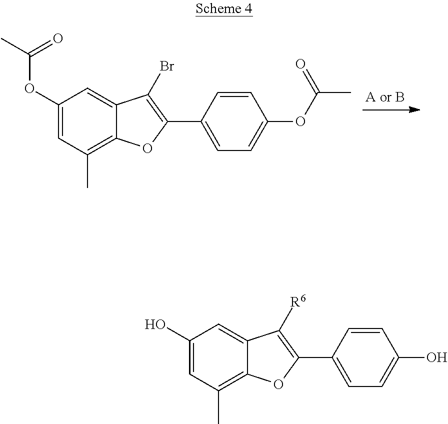

Two variants on Scheme 4 were used, as follows.

### Method A.

Boronic acids or pinacolates (0.2 mmole, 2 eq) were weighed into microwave vials. Cesium carbonate (0.2 mmole, 2 eq) was dissolved in water (326 mg/ml), and 0.2 ml of this solution was added to each vial. 2-(4-acetoxy-phenyl)-3-bromo-7-methyl-benzofuran-5-yl ester (0.1 mmole, 1 eq) was dissolved in dioxane (19.7 mg/ml) and 2 ml of this solution was added to each vial. Tetrakis(triphenylphosphine)palladium (5 mol %) was added as dry powder to each vial. Magnets were added, the vials were flushed with nitrogen, capped and run in microwave at 150 degrees for 60 minutes. The reaction mixtures were filtered and evaporated. The residues were dissolved in dry methanol (2 ml) and THF (200 ul), and treated with a few drops of 1M sodium methoxide solution for 3 hours at room temp. The reaction mixtures were neutralized with H⁺resin (Amberlyst 15) for 15 min, filtered and chromatographed on preparative reversed phase HPLC. Appropriate fractions were combined and evaporated, and identified by ¹H-NMR and LC/MS. Purity was determined by analytical HPLC.

### Method B

Acetic acid 2-(4-acetoxy-phenyl)-3-bromo-7-methyl-benzofuran-5-yl ester (30 mg, 0.074 mmol, 1 eq) was dissolved in dry DMF and dispensed into the vials. LiCl (23 mg, 0.52 mmol, 7 eq) and tetrakis (0.05 eq) were added with a spatula. The vials were again flushed with nitrogen. The tin reagents were added with a pipette (0.089 mmol, 1.2 eq). The vials were capped and run in microwave at 100 degrees for 10 minutes.

The solvents were removed by vacuum centrifugation, and the residue was dissolved in dry methanol (2 ml). A few drops of 1M sodium methoxide solution were added. The solution was stirred at room temp for 20 min, neutralized with Amberlyst 15 (H⁺resin), filtered, evaporated to 1.5 ml by applying nitrogen flow, and purified by prep HPLC using 20-50% ACN, 20 min acidic gradient.

The following Examples illustrate the invention.

### Example 1

### 2-(4-Hydroxy-phenyl)-7-methyl-3-phenyl-benzofuran-5-ol (E1)

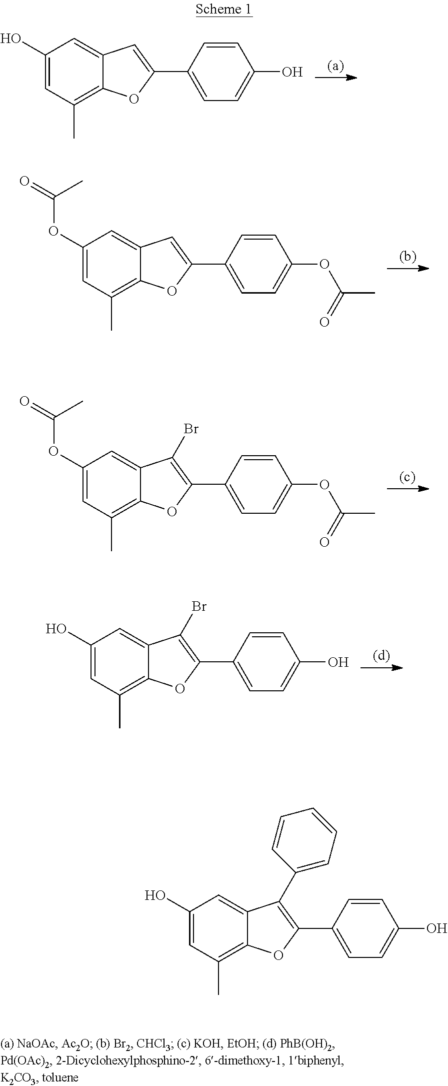

### (a) Acetic acid 2-(4-acetoxy-phenyl)-7-methyl-benzofuran-5-yl ester

2-(4-Hydroxy-phenyl)-7-methyl-benzofuran-5-ol (prepared according to WO03/51860; 16 mg, 0.067 mmol) and sodium acetate (8 mg, 0.13 mmol) were mixed with acetic anhydride (1 mL) and stirred at room temperature overnight. Methanol (1 mL) was added and the mixture was stirred for 30 minutes, then saturated aqueous sodium bicarbonate (3 mL) was added, the mixture was extracted with CH₂Cl₂ and filtered through an isolute phase separator. The organic phase was evaporated to give the title compound in quantitative yield.

### (b) Acetic acid 2-(4-acetoxy-phenyl)-3-bromo-7-methyl-benzofuran-5-yl ester

Acetic acid 2-(4-acetoxy-phenyl)-7-methyl-benzofuran-5-yl ester (24 mg, 0.067 mmol) was dissolved in CHCl₃ (1 mL), the mixture was cooled to 0° C., bromine (3.5 μL, 0.067 mmol) was added, the mixture was stirred at 0° C. for one hour, then at room temperature for 2 h. The solvent was evaporated to give the title compound in quantitative yield.

### (c) 3-Bromo-2-(4-hydroxy-phenyl)-7-methyl-benzofuran-5-ol

To a solution of acetic acid 2-(4-acetoxy-phenyl)-3-bromo-7-methyl-benzofuran-5-yl ester (2 mg, 4.5 μmol) in EtOH (0.5 mL) at room temperature was added KOH (2 mg, 0.032 mmol) and the mixture was stirred for 30 minutes. A few drops of aqueous HCl (2 M) were added to adjust the pH to 1. The mixture was diluted with water and extracted with CH₂Cl₂, then filtered through a phase separator, and the organic phase was evaporated to give the title compound in quantitative yield.

### (d) 2-(4-Hydroxy-phenyl)-7-methyl-3-phenyl-benzofuran-5-ol

A mixture of acetic acid 2-(4-acetoxy-phenyl)-3-bromo-7-methyl-benzofuran-5-yl ester (8 mg, 0.018 mmol), 2-dicyclohexylphosphino-2′,6′-dimethoxy-1-1′-biphenyl (0.4 mg, 0.001 mmol), palladium acetate (0.1 mg, 0.0004 mmol), potassium carbonate (5.2 mg, 0.038 mmol), and phenylboronic acid (4.4 mg, 0.036 mmol) in toluene (0.5 mL) was stirred at 90° C. overnight. After cooling, water and CH₂Cl₂ were added, and the mixture was filtered through a phase separator. The organic phase was evaporated and the residue was purified by preparative HPLC to give the title compound (3 mg, 52%). ES/MS m/z: 317 (M+H), 315.1 (M−H); ¹H NMR (acetone-d6, 500 MHz): 7.66-7.61 (m, 4H), 7.18 (dd, 1H, J=4.4, 1.6 Hz), 6.96 (m, 2H), 6.84 (m, 1H), 6.77 (m, 1H) and 2.52 (s, 3H).

### Example 2

### 5-Hydroxy-2-(4-hydroxy-phenyl)-3-phenyl-benzofuran-7-carbonitrile (E2)

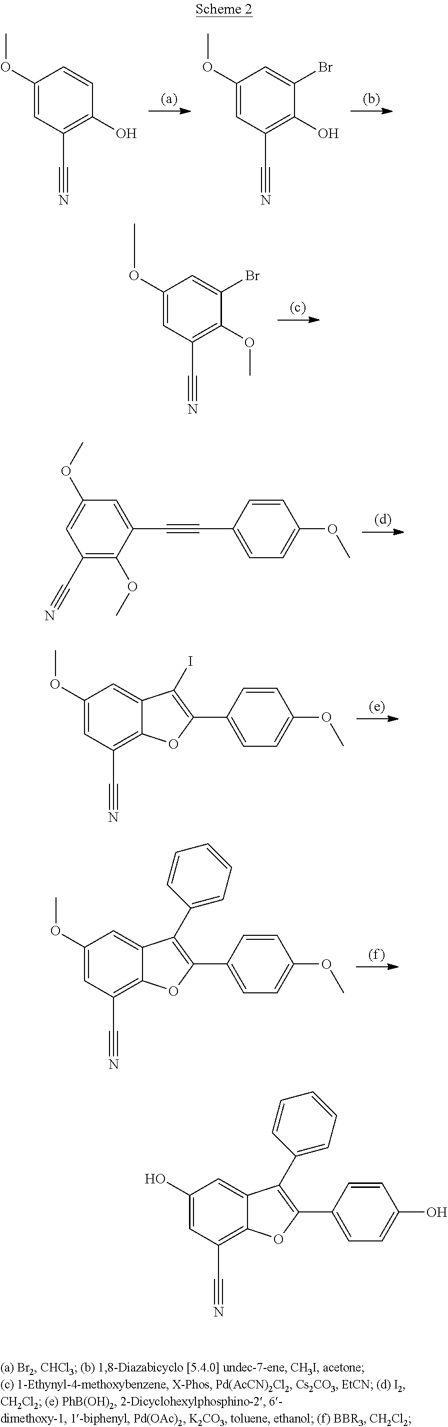

### (a) 3-Bromo-2-hydroxy-5-methoxy-benzonitrile

To an ice cold solution of 2-Hydroxy-5-methoxy-benzonitrile (1.00 g, 6.71 mmol) in CHCl₃ (50 mL) was added dropwise over a period of 30 minutes a solution of bromine (345 μL, 6.71 mmol) in CHCl₃ (50 mL) and the mixture was stirred at 0° C. for another 30 minutes. The mixture was washed with an excess of an aqueous solution of sodium bisulfite until the orange bromine color faded, and was then filtered through a phase separator. The organic phase was evaporated to give 1.53 g (100%) of the title compound.

### (b) 3-Bromo-2,5-dimethoxy-benzonitrile

To a stirred solution of 3-Bromo-2-hydroxy-5-methoxy-benzonitrile (1.53 g, 6.71 mmol) and iodomethane (4.10 mL, 65.8 mmol) in acetone (100 mL) was added dropwise over 1 h a solution of 1,8-diazabicyclo[5.4.0]undec-7-ene (3.92 mL, 26.3 mmol) in acetone (50 mL). After addition was complete the reaction mixture was stirred for another hour, then ˜80% of the solvent was evaporated, and the residue was diluted with water and extracted with CH₂Cl₂. It was then filtered through a phase separator, the organic phase was evaporated and the residue was purified by column chromatography (mobile phase:CH₂Cl₂-isohexane 1:1) to give the title compound (1.62 g, 100%).

### (c) 2,5-Dimethoxy-3-(4-methoxy-phenylethynyl)-benzonitrile

3-Bromo-2,5-dimethoxy-benzonitrile (505 mg, 2.07 mmol), dicyclohexyl-(2′,4′,6′-triisopropyl-biphenyl-2-yl)-phosphane (30 mg, 0.062 mmol), bisacetonitrile palladium (II) chloride (5 mg, 0.021 mmol), cesium carbonate (1.35 g, 4.14 mmol) and propionitrile (15 mL) were stirred at reflux under a flow of nitrogen gas. A solution of 1-ethynyl-4-methoxy-benzene (287 mg, 2.17 mmol) in propionitrile (5 mL) was added via syringe dropwise over 30 minutes and after addition was complete the mixture was stirred at reflux for an additional 2 h. After cooling and dilution with water the mixture was extracted with CH₂Cl₂, filtered through a phase separator, the organic phase was evaporated and the residue was purified by column chromatography (mobile phase:CH₂Cl₂-isohexane 1:2) to give the title compound (550 mg, 91%).

### (d) 3-Iodo-5-methoxy-2-(4-methoxy-phenyl)-benzofuran-7-carbonitrile

To a solution of 2,5-dimethoxy-3-(4-methoxy-phenylethynyl)-benzonitrile (550 mg, 1.88 mmol) in CH₂Cl₂ (15 mL) that was kept dark using aluminum foil around the reaction vessel, iodine (952 mg, 3.76 mmol) was added and the mixture was stirred at room temperature for 3 h. An excess of an aqueous solution of NaHSO₃ was added and the mixture was stirred until the violet iodine color had faded. It was then filtered through a phase separator, the organic phase was evaporated and the residue was purified by column chromatography (mobile phase:CH₂Cl₂-isohexane 1:1) to give the title compound (762 mg, 100%). Reference for the iodocyclization: J. Org. Chem. 2005, 70, 10292-20296.

### (e) 5-Methoxy-2-(4-methoxy-phenyl)-3-phenyl-benzofuran-7-carbonitrile

A mixture of 3-iodo-5-methoxy-2-(4-methoxy-phenyl)-benzofuran-7-carbonitrile (20 mg, 0.049 mmol), dicyclohexyl-(2′,6′-dimethoxy-biphenyl-2-yl)-phosphane (1.6 mg, 0.004 mmol), palladium acetate (0.23 mg, 0.001 mmol), potassium carbonate (14 mg, 0.098 mmol), phenylboronic acid (12 mg, 0.098 mmol), toluene (0.9 mL) and EtOH (0.1 mL) was stirred at room temperature overnight. The mixture was then diluted with CH₂Cl₂, washed with water, and filtered through a phase separator. The organic phase was evaporated and the residue was purified by column chromatography (mobile phase CH₂Cl₂-isohexane 1:1) to give the title compound (17 mg, 97%).

### (f) 5-Hydroxy-2-(4-hydroxy-phenyl)-3-phenyl-benzofuran-7-carbonitrile

A solution of boron tribromide in CH₂Cl₂ (1 M, 240 μL, 0.24 mmol) was added to a stirred solution of 5-methoxy-2-(4-methoxy-phenyl)-3-phenyl-benzofuran-7-carbonitrile (17 mg, 0.048 mmol) and the mixture was stirred at room temperature for 1 h. The reaction was quenched with methanol, washed with NaHCO₃ (saq), and filtered through a phase separator. The organic phase was evaporated and the residue was purified by column chromatography (mobile phase CH₂Cl₂-methanol 19:1) to give the title compound (13 mg, 83%). ES/MS m/z: 328.1 (M+H), 326.2 (M−H); ¹H NMR (MeOH-d4, 500 MHz): 7.45-7.50 (m, 2H), 7.39-7.45 (m, 5H), 7.0-7.03 (m, 2H), 6.71-6.75 (m, 2H).

### Example 3

### 2-(2-Fluoro-4-hydroxy-phenyl)-7-methyl-3-phenyl-benzofuran-5-ol (E3)

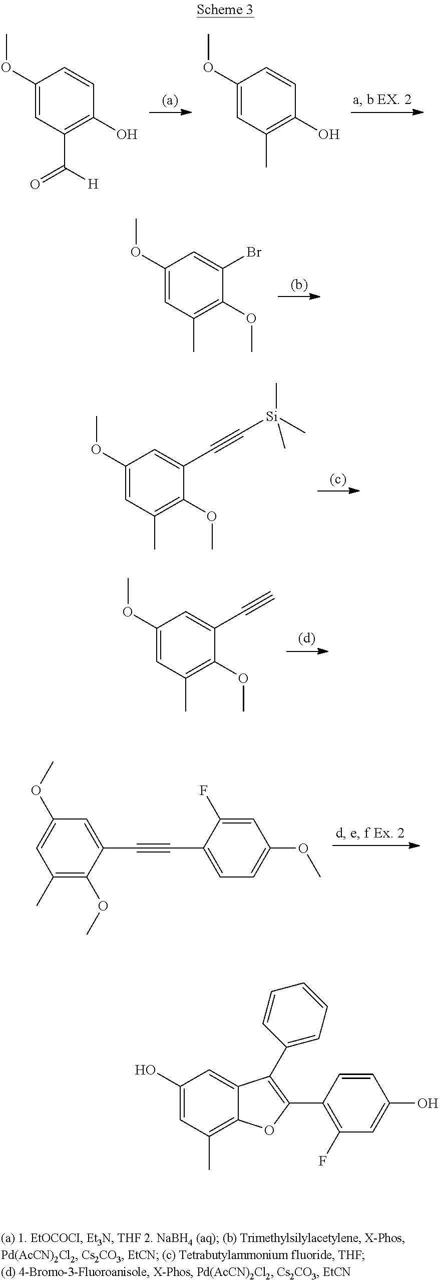

### (a) 4-Methoxy-2-methyl-phenol

The title compound was obtained from 2-hydroxy-5-methoxy-benzaldehyde using the method described in Chem Pharm Bull 27 (6), 1979, pp 1490-1494: Ethyl chloroformate (571 μL, 6.0 mmol) was added dropwise over a period of 30 minutes to an ice cold solution of 2-hydroxy-5-methoxy-benzaldehyde (624 μL, 5.0 mmol) and triethyl amine (832 μL, 6.0 mmol) in THF (5 mL) and the mixture was stirred at ° C. for 30 more minutes. The precipitate was filtered off and the filtrate was dropwise over a period of 45 minutes added to an ice cold solution of NaBH₄ (756 mg, 20 mmol) in water (7.5 mL). The resulting mixture was stirred at room temperature for 90 minutes, then diluted with water, acidified to pH<2 with 2 M HCl and extracted with ether. The organic phase was dried (MgSO₄) and evaporated. When 90% of the ether had evaporated a precipitate formed that was filtered away. The title compound was in the filtrate. Quantitative yield.

### (b) (2,5-Dimethoxy-3-methyl-phenylethynyl)-trimethyl-silane

1-Bromo-2,5-dimethoxy-3-methyl-benzene (prepared from 4-methoxy-2-methyl-phenol according to the first steps of method 2, 1.62 g, 6.61 mmol), dicyclohexyl-(2′,4′,6′-triisopropyl-biphenyl-2-yl)-phosphane (94 mg, 0.20 mmol), bisacetonitrile palladium (II) chloride (17 mg, 0.066 mmol), cesium carbonate (4.31 g, 13.2 mmol) and propionitrile (40 mL) were stirred at reflux under a flow of nitrogen gas. Ethynyl-trimethyl-silane (2.04 mL, 13.2 mmol) was added via syringe and the mixture was stirred at reflux for an additional 2 h. After cooling and dilution with water the mixture was extracted with CH₂Cl₂, and filtered through a phase separator. The organic phase was evaporated and the residue was purified by column chromatography (mobile phase:CH₂Cl₂-isohexane 1:3) to give the title compound (312 mg, 19%) along with a considerable amount of 1-Ethynyl-2,5-dimethoxy-3-methyl-benzene (242 mg, 21%).

### (c) 1-Ethynyl-2,5-dimethoxy-3-methyl-benzene

To a solution of (2,5-dimethoxy-3-methyl-phenylethynyl)-trimethyl-silane (312 mg, 1.26 mmol) in THF (10 mL) was added tetrabutylammonium fluoride hydrate (362 mg, 1.38 mmol) and the mixture was stirred at room temperature for 1 h. The mixture was diluted with ether and washed with water, the phases were separated, and the organic phase was dried (MgSO₄), filtered and evaporated. The residue was filtered through a silica plug (2 g) using CH₂Cl₂ as the eluent to give the title product in quantitative yield.

### (d) 1-(2-Fluoro-4-methoxy-phenylethynyl)-2,5-dimethoxy-3-methyl-benzene

1-Bromo-2-fluoro-4-methoxy-benzene (47 mg, 0.23 mmol), dicyclohexyl-(2′,4′,6′-triisopropyl-biphenyl-2-yl)-phosphane (3 mg, 0.0068 mmol), bisacetonitrile palladium (II) chloride (0.6 mg, 0.0023 mmol), cesium carbonate (150 mg, 0.46 mmol) and propionitrile (0.5 mL) were stirred at reflux under a flow of nitrogen gas. A solution of 1-ethynyl-2,5-dimethoxy-3-methyl-benzene (40 mg, 0.23 mmol) in propionitrile (0.5 mL) was added via syringe and the mixture was stirred at reflux for an additional 2 h. After cooling and dilution with water the mixture was extracted with CH₂Cl₂ and filtered through a phase separator. The organic phase was evaporated and the residue was purified by column chromatography (mobile phase:CH₂Cl₂-isohexane 1:1) to give the title compound (58 mg, 84%).

### 2-(2-Fluoro-4-hydroxy-phenyl)-7-methyl-3-phenyl-benzofuran-5-ol

The title compound was prepared from 1-(2-fluoro-4-methoxy-phenylethynyl)-2,5-dimethoxy-3-methyl-benzene in the three final steps described in method 2. ES/MS m/z: 353.3 (M+H), 351.4 (M−H); ¹H NMR (MeOH-d4, 500 MHz): 7.33-7.39 (m, 4H), 7.26-7.31 (m, 3H), 6.80 (d, 2.23 Hz, 1H), 6.64-6.66 (m, 1H), 6.62 (dd, 2.24 Hz, 8.56 Hz, 1H), 6.52 (dd, 2.50 Hz, 12.00 Hz, 1H), 2.48 (s, 3H).

### Example 4

### 7-Dibromomethyl-2-(4-hydroxy-phenyl)-3-phenyl-benzofuran-5-ol (E4)

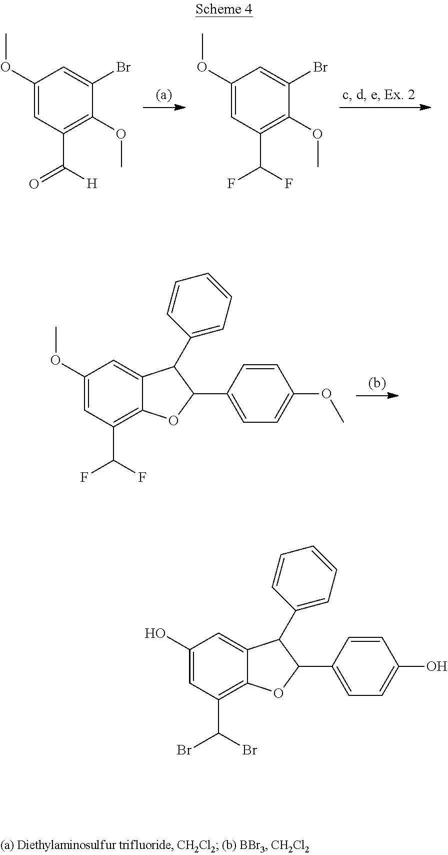

### (a) 1-Bromo-3-difluoromethyl-2,5-dimethoxy-benzene

To a plastic tube containing a solution of 3-bromo-2,5-dimethoxy-benzaldehyde (prepared from 2-hydroxy-5-methoxy-benzaldehyde according to the first steps of method 2, 108 mg, 0.44 mmol) in CH₂Cl₂ (3 mL) was added diethylaminosulfur trifluoride (291 μL, 2.2 mmol) and the mixture was stirred at 40° C. overnight. After cooling the mixture was poured into water, filtered through a phase separator. The organic phase was evaporated and the residue was purified by column chromatography (mobile phase:CH₂Cl₂-isohexane 1:3 to give the title compound (96 mg, 82%).

### (b) 7-Dibromomethyl-2-(4-hydroxy-phenyl)-3-phenyl-benzofuran-5-ol

A solution of boron tribromide in CH₂Cl₂ (1 M, 220 μL, 0.22 mmol) was added to a stirred solution of 7-difluoromethyl-5-methoxy-2-(4-methoxy-phenyl)-3-phenyl-benzofuran (prepared from 1-bromo-3-difluoromethyl-2,5-dimethoxy-benzene according to method 2, 17 mg, 0.045 mmol) and the mixture was stirred at room temperature for 1 h. The reaction was quenched with methanol, washed with NaHCO₃ (saq), and filtered through a phase separator. The organic phase was evaporated and the residue was purified by column chromatography (mobile phase CH₂Cl₂-methanol 19:1) to give the title compound (5 mg, 23%). ¹H NMR (CDCl₃, 500 MHz): 7.58-7.61 (m, 2H), 7.43-7.49 (m, 4H), 7.38-7.42 (m, 1H), 7.22 (s, 1H), 7.19 (d, 2.46 Hz, 1H), 6.85-6.89 (m, 3H).

### Example 5

### [5-Hydroxy-2-(4-hydroxy-phenyl)-3-phenyl-benzofuran-7-yl]-acetonitrile (E5)

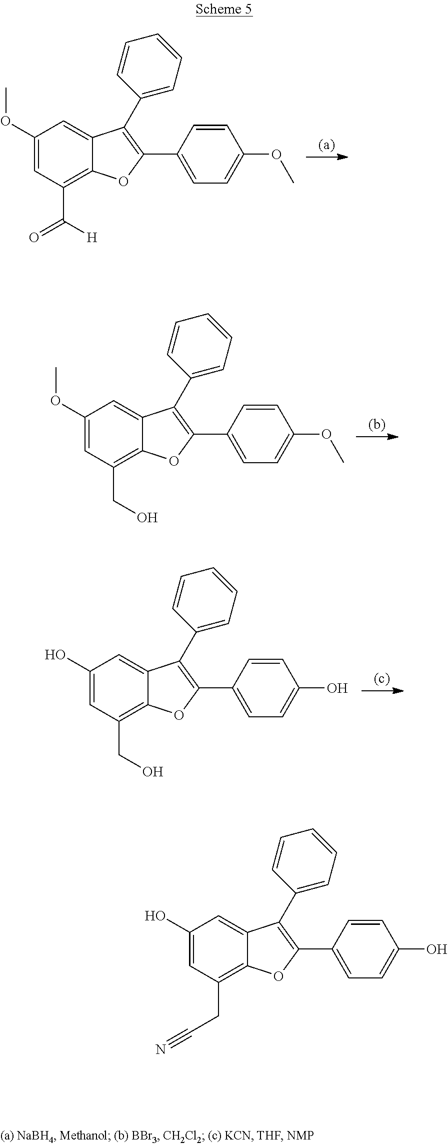

### (a) [5-Methoxy-2-(4-methoxy-phenyl)-3-phenyl-benzofuran-7-yl]-methanol

To a solution of 5-methoxy-2-(4-methoxy-phenyl)-3-phenylbenzofuran-7-carbaldehyde (prepared from 2-hydroxy-5-methoxy-benzaldehyde according to method 2, 53 mg, 0.15 mmol) in methanol (3 mL) was added NaBH₄ (11 mg, 0.30 mmol) and the mixture was stirred at room temperature for 1 h. The reaction was quenched with 1 M HCl (aq) and the mixture was diluted with water. The title compound precipitated and was isolated by filtration. Yield: 50 mg (94%).

### (b) 7-Bromomethyl-2-(4-hydroxy-phenyl)-3-phenyl-benzofuran-5-ol

To an ice cold solution of [5-methoxy-2-(4-methoxy-phenyl)-3-phenyl-benzofuran-7-yl]-methanol (50 mg, 0.14 mmol) in CH₂Cl₂ (3 mL) was added a 1 M solution of BBr₃ in CH₂Cl₂ (0.56 mL, 0.56 mmol) and the mixture was stirred at 0° C. for 4 h. The reaction was quenched by the addition of methanol, washed with sodium bicarbonate (saq), and filtered through a phase separator. The organic phase was evaporated and the residue was purified by column chromatography (mobile phase:CH₂Cl₂-methanol 39:1) to give the title compound (26 mg, 44%).

### (c) [5-Hydroxy-2-(4-hydroxy-phenyl)-3-phenyl-benzofuran-7-yl]-acetonitrile

Potassium cyanide (3 mg, 0.039 mmol) and 7-bromomethyl-2-(4-hydroxy-phenyl)-3-phenyl-benzofuran-5-ol (14 mg, 0.035 mmol) were suspended in a 3:1 mixture of THF and NMP (0.5 mL) and the mixture was stirred at 60° C. overnight. Sodium bicarbonate (saq) was added and the mixture was extracted with a 4:1 mixture of CH₂Cl₂ and methanol, and filtered through a phase separator. The organic phase was evaporated and the residue was purified by column chromatography (mobile phase:CH₂Cl₂-methanol 39:1) to give the title compound (2 mg, 17%). ES/MS m/z: 342.2 (M+H), 340 (M−H); ¹H NMR: (MeOH-d4, 500 MHz): 7.38-7.50 (m, 7H), 6.83 (d, 2.37 Hz, 1H), 6.75 (d, 2.37 Hz, 1H), 6.71-6.74 (m, 2H), 4.16 (s, 2H).

### Example 6

### 2-(4-Hydroxy-phenyl)-7-(1-methoxy-ethyl)-3-phenyl-benzofuran-5-ol (E6)

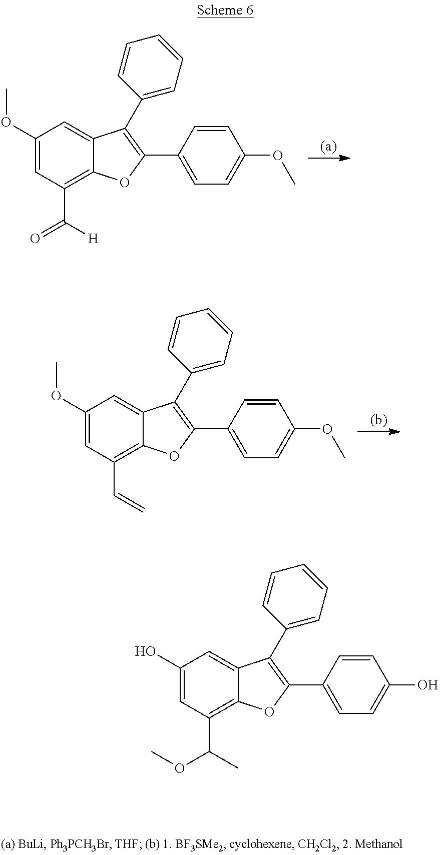

### (a) 5-Methoxy-2-(4-methoxy-phenyl)-3-phenyl-7-vinyl-benzofuran

Butyllithium (2.5 M in hexanes, 0.08 mL, 0.2 mmol) was added to an ice cold slurry of methyltriphenylphosphinium bromide in THF (1 mL). The mixture was stirred at 0° C. for 15 minutes. 5-Methoxy-2-(4-methoxy-phenyl)-3-phenylbenzofuran-7-carbaldehyde (36 mg, 0.10 mmol) was added and the mixture was stirred at 50° C. for three hours. The reaction was quenched by the careful addition of 2M HCl, extracted with CH₂Cl₂, filtered through a phase separator, the organic phase was evaporated and the residue was purified by column chromatography (mobile phase:CH₂Cl₂-isohexane 1:2) to give the title compound (20 mg, 89%).

### (b) 2-(4-Hydroxy-phenyl)-7-(1-methoxy-ethyl)-3-phenyl-benzofuran-5-ol

5-Methoxy-2-(4-methoxy-phenyl)-3-phenyl-7-vinyl-benzofuran (12 mg, 0.034 mmol) and cyclohexene (34 μL, 0.34 mmol) were dissolved in CH₂Cl₂ (1 mL) and stirred at room temperature. BF₃.S(CH₃)₂ (14 μL, 0.135 mmol) was added and the mixture was stirred at room temperature overnight. Methanol was added, followed by NaHCO₃ (saq). After filtration through a phase separator the organic phase was evaporated and the residue was purified by column chromatography (mobile phase:CH₂Cl₂-methanol 39:1) to give the title compound (2 mg, 15%). ES/MS m/z: 361.4 (M+H), 359.2 (M−H); ¹H NMR: (MeOH-d4, 500 MHz): 7.37-7.50 (m, 7H), 6.78 (d, 2.43 Hz, 1H), 6.70-6.74 (m, 2H), 6.69 (d, 2.43 Hz, 1H), 4.91 (q, 6.55 Hz, 1H), 3.34 (s, 1H), 1.60 (d, 6.55 Hz, 3H).

### Example 7

### 7-Difluoromethyl-2-(4-hydroxy-phenyl)-3-phenyl-benzofuran-5-ol (E7)

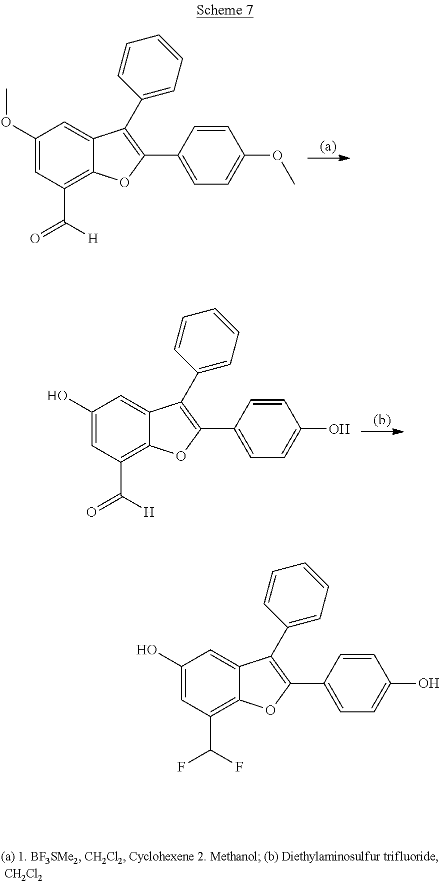

### (a) 5-Hydroxy-2-(4-hydroxy-phenyl)-3-phenyl-benzofuran-7-carbaldehyde

5-Methoxy-2-(4-methoxy-phenyl)-3-phenylbenzofuran-7-carbaldehyde (80 mg, 0.22 mmol) and cyclohexene (220 μL, 2.2 mmol) were dissolved in CH₂Cl₂ (3 mL) and stirred at room temperature. BF₃.S(CH₃)₂ (56 μL, 0.54 mmol) was added and the mixture was stirred at room temperature overnight. Methanol was added, followed by NaHCO₃ (saq). After filtration through a phase separator the organic phase was evaporated and the residue was purified by column chromatography (mobile phase:CH₂Cl₂-methanol 39:1) to give the title compound (20 mg, 27%).

### (b) 7-Difluoromethyl-2-(4-hydroxy-phenyl)-3-phenyl-benzofuran-5-ol

A solution of 5-hydroxy-2-(4-hydroxy-phenyl)-3-phenyl-benzofuran-7-carbaldehyde (15 mg, 0.045 mmol) and diethylaminosulfur trifluoride (22 μL, 0.18 mmol) in CH₂Cl₂ (0.5 mL) was stirred overnight at 40° C. in a plastic tube. After cooling, the reaction was quenched by the addition of methanol and the mixture was washed with NaHCO₃ (saq). After filtration through a phase separator the organic phak was evaporated and the residue was purified by column chromatography (mobile phase:CH₂Cl₂-methanol 19:1) to give the title compound (5 mg, 31%). ES/MS m/z: 353.3 (M+H), 351.3 (M−H); ¹H NMR: (MeOH-d4, 500 MHz): 7.39-7.51 (m, 7H), 7.18 (t, 55.13 Hz, 1H), 6.93-6.95 (m, 1H), 6.87-6.89 (m, 1H), 6.71-6.75 (m, 2H).

### Example 8

### 2-(4-Hydroxy-phenyl)-3-phenyl-7-vinyl-benzofuran-5-ol (8)

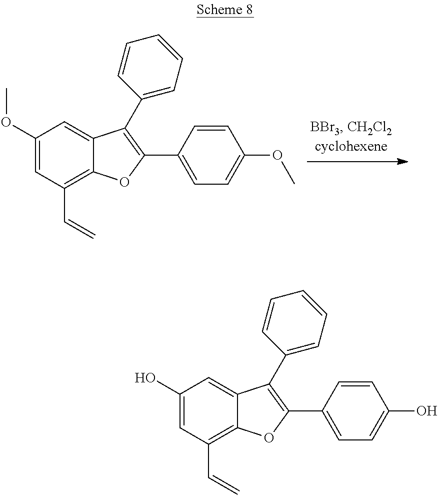

### 2-(4-Hydroxy-phenyl)-3-phenyl-7-vinyl-benzofuran-5-ol

To a solution of 5-methoxy-2-(4-methoxy-phenyl)-3-phenyl-7-vinyl-benzofuran (prepared according to method 6, 4 mg, 0.011 mmol) and cyclohexene (23 μL, 0.22 mmol) was added BBr₃ (1 M in CH₂Cl₂, 56 μL, 0.056 mmol) at −78° C. The mixture was then stored in the freezer (−18° C.) overnight. The reaction was quenched with water, washed with NaHCO₃ (saq), and filtered through a phase separator. The organic phase was evaporated and the residue was purified by column chromatography (mobile phase:CH₂Cl₂-methanol 39:1) to give the title compound (2 mg, 54%). ES/MS m/z: 329.1 (M+H), 327.2 (M−H); ¹H NMR: (MeOH-d4, 500 MHz): 7.38-7.50 (m, 7H), 6.97 (dd, 13.37 Hz, 17.76 Hz, 1H), 6.81 (d, 2.52 Hz, 1H), 6.71-6.75 (m, 2H), 6.69 (d, 2.52 Hz, 1H), 6.25 (dd, 1.35 Hz, 17.76 Hz, 1H), 5.52 (dd, 1.35 Hz, 11.37 Hz, 1H).

### Example 9

### 2-(4-Hydroxy-phenyl)-3-thiophen-3-yl-7-trifluoromethyl-benzofuran-5-ol (E9)

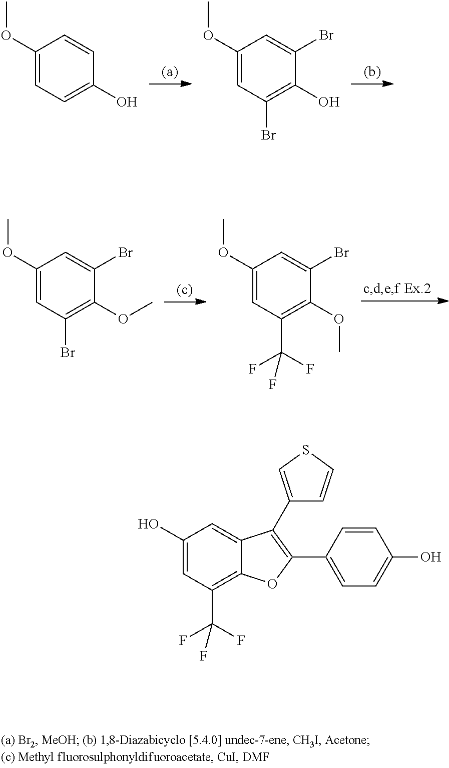

### (a) 2,6-Dibromo-4-methoxy-phenol

A solution of 4-methoxyphenol (5.72 g, 46.1 mmol) in MeOH (80 mL) was cooled to 0° C. while stirring. A solution of Br₂ (4.97 mL, 96.8 mmol) in MeOH (20 mL) was added dropwise and the resulting mixture was stirred at room temperature for 3 h. The reaction was quenched by the addition of aqueous solutions of NaHCO₃ and NaHSO₃. When gas evolution ceased and the bromine color had faded, water was added and a thick yellowish precipitate was formed. The precipitate was filtered off and recrystallized from MeOH—H₂O to give the title product (5.20 g, 40%).

### (b) 1,3-Dibromo-2,5-dimethoxy-benzene

To a stirred solution of 2,6-dibromo-4-methoxy-phenol (5.2 g, 18.4 mmol) and CH₃I (11.5 mL, 184 mmol) in dry acetone (50 mL) was added a solution of 1,8-diazabicyclo[5.4.0]undec-7-ene (13.8 mL, 92.2 mmol) in dry acetone (50 mL) dropwise during 30 min. When the addition was complete the reaction mixture was stirred at room temp for 30 minutes. The mixture was poured into 2 M HCl (aq) and extracted with ether. The organic phase was dried, filtered and evaporated and the residue was purified by column chromatography (mobile phase CH₂Cl₂-isohexane 9:1→1:1) to give the desired bisether as a colorless liquid.

### (c) 1-Bromo-2,5-dimethoxy-3-trifluoromethyl-benzene

To a stirred solution of 1,3-dibromo-2,5-dimethoxy-benzene (353 mg, 1.19 mmol) and difluoro-fluorosulfonyl-acetic acid methyl ester (455 μL, 3.58 mmol) in DMF (1 mL) under N₂ was added CuI (227 mg, 1.19 mmol). The mixture was then stirred at 80° C. over three days under N₂. The reaction was worked up by cooling, dilution with CH₂Cl₂, washing with water, and filtering through a phase separator. The organic phase was evaporated and the residue was purified by column chromatography (mobile phase CH₂Cl₂-isohexane 1:9→1:1) to give the title compound (60 mg, 18%).

### 2-(4-Hydroxy-phenyl)-3-thiophen-3-yl-7-trifluoromethyl-benzofuran-5-ol

The title compound was prepared from 1-bromo-2,5-dimethoxy-3-trifluoromethyl-benzene in the four final steps described in example 2. ES/MS m/z: 377.2 (M+H), 375.2 (M−H).

### Example 10

### 7-Fluoro-2-(1H-indazol-5-yl)-3-thiophen-3-yl-benzofuran-5-ol (E10)

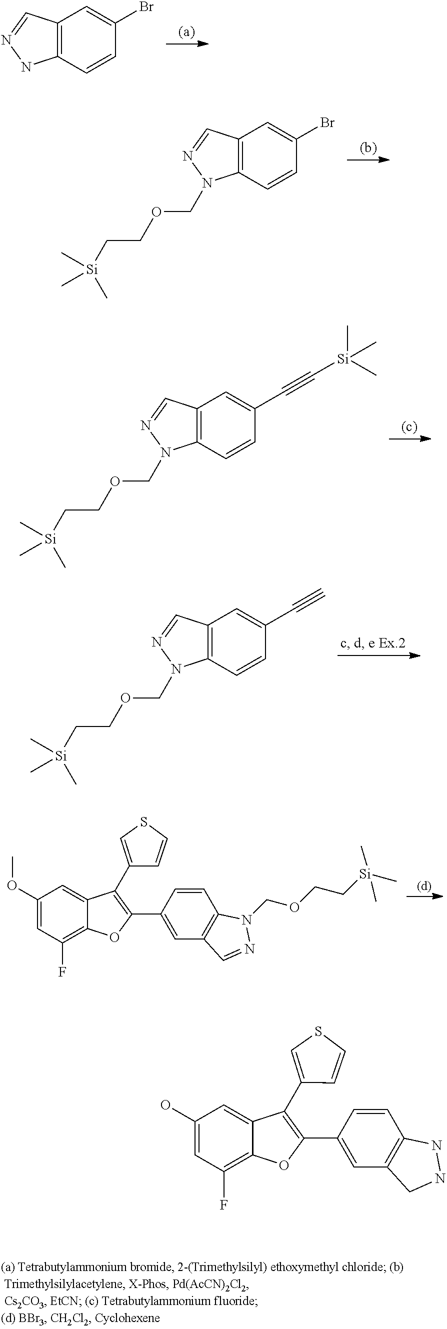

### (a) 5-Bromo-1-(2-trimethylsilanyl-ethoxymethyl)-1H-indazole

A mixture of the 5-bromo-1H-indazole (2.00 g, 10.15 mmol), tetrabutylammonium bromide (33 mg, 0.10 mmol) and 2 M NaOH (aq, 10.15 mL, 20.3 mmol) in CH₂Cl₂ (30 mL) was cooled in an ice bath, a solution of (2-chloromethoxy-ethyl)-trimethyl-silane in CH₂Cl₂ (20 mL) was added dropwise during 30 minutes and the mixture was stirred at 0° C. for 1 h then allowed to warm to room temperature and stirred for one additional hour. The mixture was filtered through a phase separator, the organic phase was evaporated to give the title compound and the regioisomeric 5-bromo-2-(2-trimethylsilanyl-ethoxymethyl)-2H-indazole in a 3:1 ratio in a combined quantitative yield.

### (b) 1-(2-Trimethylsilanyl-ethoxymethyl)-5-trimethylsilanylethynyl-1H-indazole

A mixture of 5-bromo-1-(2-trimethylsilanyl-ethoxymethyl)-1H-indazole (3.32 g, 10.15 mmol), dicyclohexyl-(2′,4′,6′-triisopropyl-biphenyl-2-yl)-phosphane (387 mg, 0.81 mmol), bisacetonitrile palladium (II) chloride (53 mg, 0.20 mmol), cesium carbonate (6.61 g, 20.30 mmol), and propionitrile (60 mL) was stirred at to 80° C. under argon. Ethynyl-trimethyl-silane (4.3 mL, 30.5 mmol) was added via syringe and the mixture was stirred at 80° C. for 2 h. The mixture was cooled, diluted with water and ether; the phases were separated, the organic phase was dried, filtered, and evaporated and the residue was purified by column chromatography (mobile phase:CH₂Cl₂-isohexane 1:9→1:0) to give the title compound (3.10 g, 89%).

### (c) 5-Ethynyl-1-(2-trimethylsilanyl-ethoxymethyl)-1H-indazole

1-(2-Trimethylsilanyl-ethoxymethyl)-5-trimethylsilanylethynyl-1H-indazole (357 mg, 1.04 mmol), tetrabutylammonium fluoride hydrate (271 mg, 1.04 mmol), and acetonitrile (10 mL) were mixed and stirred at 0° C. for 10 minutes. The solvent was evaporated and the residue was filtered through a 5 g silica plug. The title compound (235 mmol, 83%) was eluted with a mixture of CH₂Cl₂ and isohexane (1:1).

### (d) 7-Fluoro-2-(1H-indazol-5-yl)-3-thiophen-3-yl-benzofuran-5-ol

To a solution of 5-(7-fluoro-5-methoxy-3-thiophen-3-yl-benzofuran-2-yl)-1-(2-trimethylsilanyl-ethoxymethyl)-1H-indazole (prepared from 2-fluoro-4-methoxy-phenol and eventually 5-ethynyl-1-(2-trimethylsilanyl-ethoxymethyl)-1H-indazole according to method 2, 20 mg, 0.04 mmol) and cyclohexene (82 μL, 0.81 mmol) in CH₂Cl₂ (1 mL) at 6° C. was added BBr₃ (1 M in CH₂Cl₂, 0.40 mL, 0.40 mmol) and the mixture was left at that temperature overnight. The reaction was quenched with water, pH was adjusted to ˜10 with sodium carbonate (saq), extracted with EtOAc, the phases were separated, the organic phase was dried (Na₂CO₃), filtered, and evaporated. The residue was purified by column chromatography (mobile phase: CH₂Cl₂-methanol 99:1→19:1) to give the title compound (4.3 mg, 30%). ES/MS m/z: 351.1 (M+1), 349.1 (M−1); NMR (MeOH-d4, 500 MHz): 8.09-8.10 (m, 1H), 8.06 (d, 093 Hz, 1H), 7.63 (dd, 1.52 Hz, 8.81 Hz, 1H), 7.58 (dd, 2.83 Hz, 5.04 Hz, 1H), 7.53 (dd, 1.27 Hz, 3.14 Hz, 1H), 7.49-7.52 (m, 1H), 7.12 (dd, 1.28 Hz, 5.03 Hz, 1H), 6.69 (d, 2.14 Hz, 1H), 6.61 (dd, 2.14 Hz, 12.05 Hz, 1H).

### Example 11

### 2-[7-Chloro-5-hydroxy-2-(4-hydroxy-phenyl)-benzofuran-3-yl]-furan-3-carbonitrile (E11)

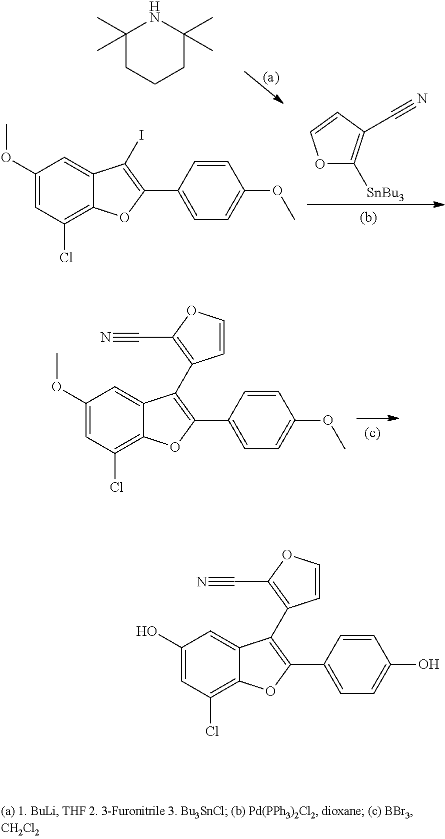

### (a) 2-Tributylstannanyl-furan-3-carbonitrile

Butyllithium (1.6 M in hexanes, 3.3 mL, 5.24 mmol) was added to an ice cold solution of 2,2,6,6-tetramethylpiperidine (1.00 mL, 5.90 mmol) in THF (10 mL). The solution was stirred at 0° C. for 30 minutes, then cooled to −78° C. A solution of furan-3-carbonitrile (610 mg, 6.56 mmol) in THF (10 mL) was added dropwise over 30 minutes and the mixture was stirred for 2 h at −78° C. A solution of tributyltin chloride in THF (2 mL) was added dropwise over 30 minutes and the mixture was stirred overnight. The temperature was allowed to rise to room temperature during that time. The reaction was quenched by the addition of NH₄Cl (saq), extracted with CHCl₃, filtered through a phase separator. The organic phase was evaporated and the residue was purified by column chromatography (mobile phase CH₂Cl₂-isohexane 0:1→1:1) to give the title compound (992 mg, 50%).

### (b) 3-[7-Chloro-5-methoxy-2-(4-methoxy-phenyl)-benzofuran-3-yl]-furan-2-carbonitrile

7-Chloro-3-iodo-5-methoxy-2-(4-methoxy-phenyl)-benzofuran (prepared from 2-chloro-4-methoxy-phenol according to method 2, 24 mg, 0.06 mmol), 2-tributylstannanyl-furan-3-carbonitrile (29 mg, 0.08 mmol) and Pd(PPh₃)₂Cl₂ (4 mg, 0.01 mmol) was mixed with dioxane (0.5 mL) under nitrogen in a microwave vial. The reaction was run in a microwave reactor at 130° C. for 40 min. NH₄Cl (saq), was added, and the mixture was extracted with CHCl₃ and filtered through a phase separator. The organic phase was evaporated and the residue was purified by column chromatography (mobile phase:CH₂Cl₂-isohexane 1:3→1:0) to give the title compound (17 mg, 77%).

### (c) 3-[7-Chloro-5-hydroxy-2-(4-hydroxy-phenyl)-benzofuran-3-yl]-furan-2-carbonitrile

To an ice cold solution of 3-[7-chloro-5-methoxy-2-(4-methoxy-phenyl)-benzofuran-3-yl]-furan-2-carbonitrile (17 mg, 0.04 mmol) in CH₂Cl₂ (1 mL) was added BBr₃ (1 M in CH₂Cl₂, 0.45 mL, 0.45 mmol). The mixture was stored in the refrigerator at 6° C. overnight without stirring. The reaction was quenched by the addition of NaHCO₃ (saq), extracted with CH₂Cl₂-MeOH19:1, and filtered through a phase separator. The organic phase was evaporated and the residue was purified by column chromatography (mobile phase CH₂Cl₂-MeOH 1:0→9:1) to give the title compound (1 mg, 6%). ES/MS m/z: 352 (M+1), 350 (M−1); ¹H NMR (MeOH-d4, 500 MHz): 7.85 (d, 2.02 Hz, 1H), 7.71 (d, 8.61 Hz, 1H), 7.44-7.49 (m, 2H), 6.93 (d, 2.00 Hz, 1H), 6.83-6.90 (m, 3H).

The following example compounds were synthesized using the procedures described above.

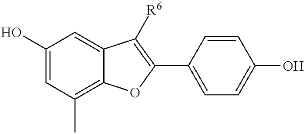

### E 12 2-(4-Hydroxy-phenyl)-7-methyl-3-thiophen-3-yl-benzofuran-5-ol

R⁶=thiophen-3-yl

ES/MS m/z: 323.6 (pos. M+H), 321.6 (neg. M−H); ¹H NMR (acetone-d6, 500 MHz): 7.66-7.61 (m, 4H), 7.18 (dd, 1H, J=4.4, 1.6 Hz), 6.96 (m, 2H), 6.84 (m, 1H), 6.77 (m, 1H) and 2.52 (s, 3H).

### E 13 3-(2,5-Difluoro-phenyl)-2-(4-hydroxy-phenyl)-7-methyl-benzofuran-5-ol

R⁶=2,5-difluoro-phenyl

ES/MS m/z: 353.7 (pos. M+H), 351.7 (neg. M−H); ¹H NMR (acetone-d6, 500 MHz): 7.57 (m, 2H), 7.36 (m, 1H), 7.31 (m, 1H), 6.97 (m, 2H), 6.80 (m, 1H), 6.70 (m, 1H) and 2.55 (s, 3H).

### E 14 3-[5-Hydroxy-2-(4-hydroxy-phenyl)-7-methyl-benzofuran-3-yl]-benzonitrile

R⁶=3-cyano-phenyl

ES/MS m/z: 342.7 (pos. M+H), 340.7 (neg. M−H); ¹H NMR (acetone-d6, 500 MHz): 7.89 (m, 1H), 7.85-7.81 (m, 2H), 7.73 (m, 1H), 7.54 (m, 2H), 6.96 (m, 2H), 6.80 (m, 2H) and 2.54 (s, 3H).

### E 15 2-(4-Hydroxy-phenyl)-7-methyl-3-m-tolyl-benzofuran-5-ol

R⁶=3-methyl-phenyl

ES/MS m/z: 331.7 (pos. M+H), 329.7 (neg. M−H); ¹H NMR (acetone-d6, 500 MHz): 7.58 (m, 2H), 7.39 (t, 1H, J=7.7 Hz), 7.32 (m, 1H), 7.26 (m, 1H), 6.92 (m, 2H), 6.77 (m, 1H), 6.73 (m, 1H), 2.54 (s, 3H) and 2.38 (s, 3H).

### E 16 2-(4-Hydroxy-phenyl)-7-methyl-3-thiophen-2-yl-benzofuran-5-ol

R⁶=thiophen-2-yl

ES/MS m/z: 321.6 (neg. M−H); ¹H NMR (acetone-d6, 500 MHz): 7.66 (m, 2H), 7.61 (dd, 1H, J=4.8, 1.5 Hz), 7.24-7.21 (m, 2H), 6.97 (m, 2H), 6.84 (m, 1H), 6.79 (m, 1H) and 2.53 (s, 3H).

### E 17 2-(4-Hydroxy-phenyl)-7-methyl-3-pyridin-3-yl-benzofuran-5-ol

R⁶=pyridin-3-yl

ES/MS m/z: 318.7 (neg. M+H); ¹H NMR (acetone-d6, 500 MHz): 8.68 (dd, 1H, J=2.3, 0.9 Hz), 8.63 (dd, 1H, J=5.0, 1.7 Hz), 7.90 (m, 1H), 7.55 (m, 2H), 7.51 (m, 1H), 6.96 (m, 2H), 6.80 (m, 1H), 6.78 (m, 1H) and 2.55 (s, 3H).

### E 18 2-[5-Hydroxy-2-(4-hydroxy-phenyl)-7-methyl-benzofuran-3-yl]-benzonitrile

R⁶=2-cyano-phenyl

ES/MS m/z: 342.3 (pos. M+H), 340.2 (neg. M−H); ¹H NMR (acetone-d6, 500 MHz): 7.96 (m, 1H), 7.89 (m 1H), 7.71 (m, 1H), 7.47 (m, 2H), 7.05 (m, 1H), 6.94 (m, 2H), 6.81 (m, 1H), 6.63 (m, 1H) and 2.57 (s, 3H).

### E 19 2-(4-Hydroxy-phenyl)-7-methyl-3-(3-nitro-phenyl)-benzofuran-5-ol

R⁶=3-nitro-phenyl

ES/MS m/z: 362.0 (pos. M+H), 360.0 (neg. M−H); ¹H NMR (acetone-d6, 500 MHz): 8.33 (m, 1H), 8.27 (m, 1H), 7.91 (m, 1H), 7.80 (dd, 1H, J=7.6, 8.1 Hz), 7.50 (m, 2H), 6.87 (m, 2H), 6.75-6.73 (m, 2H) and 2.52 (s, 3H).

### E 20 5-[5-Hydroxy-2-(4-hydroxy-phenyl)-7-methyl-benzofuran-3-yl]-thiophene-2-carbaldehyde

R⁶=5-thiophene-2-carbaldehyde

ES/MS m/z: 349.0 (neg. M−H); ¹H NMR (acetone-d6, 500 MHz): 9.98 (s, 1H), 8.02 (d, 1H, J=3.9 Hz), 7.60 (m, 2H), 7.38 (d, 1H, J=3.9 Hz), 6.94 (m, 2H), 6.92 (m, 1H), 6.75 (m, 1H) and 2.50 (s, 3H).

### E 21 3-(3,5-Difluoro-phenyl)-2-(4-hydroxy-phenyl)-7-methyl-benzofuran-5-ol

R⁶=3,5-difluoro-phenyl

ES/MS m/z: 351.0 (neg. M−H); ¹H NMR (acetone-d6, 500 MHz): 7.50 (m, 2H), 7.11-7.04 (m, 3H), 6.89 (m, 2H), 6.73-6.71 (m, 2H) and 2.50 (s, 3H).

### E 22 3-(3,5-Dichloro-phenyl)-2-(4-hydroxy-phenyl)-7-methyl-benzofuran-5-ol

R⁶=3,5-dichloro-phenyl

ES/MS m/z: 383+385 (neg. M−H); ¹H NMR (acetone-d6, 500 MHz): 7.52 (t, 1H, J=1.9 Hz), 7.50 (m, 2H), 7.45 (m, 2H), 6.90 (m, 2H), 6.72-6.70 (m, 2H) and 2.50 (s, 3H).

### E 23 2-(4-Hydroxy-phenyl)-7-methyl-3-(2-phenoxy-phenyl)-benzofuran-5-ol

R⁶=2-phenoxy-phenyl

ES/MS m/z: 407.0 (neg. M−H); ¹H NMR (acetone-d6, 500 MHz): 7.51 (m, 2H), 7.49-7.44 (m, 2H), 7.28 (m, 1H), 7.21-7.17 (m, 2H), 7.04 (m, 1H), 6.96 (m, 1H), 6.84 (m, 2H), 6.76 (m, 2H), 6.65 (m, 1H), 6.63 (m, 1H) and 2.45 (s, 3H).

### E 24 3-Biphenyl-2-yl-2-(4-hydroxy-phenyl)-7-methyl-benzofuran-5-ol

R⁶=biphenyl-2yl

ES/MS m/z: 391.0 (neg. M−H); ¹H NMR (acetone-d6, 500 MHz): 7.56 (m, 1H), 7.52-7.49 (m, 2H), 7.45 (m, 1H), 7.32 (m, 2H), 7.13-7.10 (m, 2H), 7.06-7.04 (m, 3H), 6.75 (m, 2H), 6.58 (m, 1H), 6.39 (m, 1H) and 2.43 (s, 3H).

### E 25 3-(2-Hydroxy-phenyl)-2-(4-hydroxy-phenyl)-7-methyl-benzofuran-5-ol

R⁶=2-hydroxy-phenyl

ES/MS m/z: 331.0 (neg. M−H); ¹H NMR (acetone-d6, 500 MHz): 7.50 (m, 2H), 7.29 (m, 1H), 7.23 (dd, 1H, J=7.6, 1.6 Hz), 7.04 (dd, 1H, J=8.3, 1.0 Hz), 6.95 (m, 1H), 6.78 (m, 2H), 6.63 (m, 1H), 6.48 (m, 1H) and 2.48 (s, 3H).

### E 26 1-{3-[5-Hydroxy-2-(4-hydroxy-phenyl)-7-methyl-benzofuran-3-yl]-phenyl}-ethanone

R⁶=4-Acetyl-phenyl

ES/MS m/z: 357.0 (neg. M−H); ¹H NMR (acetone-d6, 500 MHz): 8.10 (m, 1H), 8.02 (m, 1H), 7.70 (m, 1H), 7.64 (m, 1H), 7.49 (m, 2H), 6.84 (m, 2H), 6.71-6.70 (m, 2H), 2.60 (s, 3H) and 2.51 (s, 3H).

### E 27 2-(4-Hydroxy-phenyl)-3-(3-methanesulfonyl-phenyl)-7-methyl-benzofuran-5-ol

R⁶=3-methanesulfonyl-phenyl

ES/MS m/z: 393.0 (neg. M−H); ¹H NMR (acetone-d6, 500 MHz): 8.05 (m, 1H), 7.97 (m, 1H), 7.81 (m, 1H), 7.77 (m, 1H), 7.49 (m, 2H), 6.87 (m, 2H), 6.74-6.72 (m, 2H), 3.17 (s, 3H) and 2.51 (s, 3H).

### E 28 3-(3-Ethylsulfanyl-phenyl)-2-(4-hydroxy-phenyl)-7-methyl-benzofuran-5-ol

R⁶=3-ethylsulfanyl-phenyl

ES/MS m/z: 375.0 (neg. M−H); ¹H NMR (acetone-d6, 500 MHz): 7.50 (m, 2H), 7.44 (m, 1H), 7.41 (m, 1H), 7.36 (m, 1H), 7.28 (m, 1H), 6.85 (m, 2H), 6.69 (m, 2H), 2.97 (q, 2H, J=7.3 Hz), 2.50 (s, 3H) and 1.27 (t, 3H, J=7.3 Hz).

### E 29 2-(4-Hydroxy-phenyl)-7-methyl-3-quinolin-5-yl-benzofuran-5-ol

R⁶=quinolin-5-yl

ES/MS m/z: (neg. M−H); ¹H NMR (acetone-d6, 500 MHz): 8.94 (dd, 1H, J=4.1, 1.6 Hz), 8.18 (m, 1H), 8.04 (m, 1H), 7.90 (dd, 1H, J=8.5, 7.1 Hz), 7.66 (dd, 1H, J=7.1, 1.2 Hz), 7.39 (dd, 1H, J=8.5, 4.1 Hz), 7.33 (m, 2H), 6.72-6.69 (m, 3H), 6.28 (d, 1H, J=2.0 Hz) and 2.57 (s, 3H).

### E 30 1-{5-[5-Hydroxy-2-(4-hydroxy-phenyl)-7-methyl-benzofuran-3-yl]-thiophen-2-yl}-ethanone

R⁶=4-Acetyl-thiophene-2-yl

ES/MS m/z: 363.0 (neg. M−H); ¹H NMR (acetone-d6, 500 MHz): 7.90 (d, 1H, J=3.8 Hz), 7.60 (m, 2H), 7.26 (d, 1H, J=3.8 Hz), 6.92 (m, 2H), 6.90 (m, 1H), 6.73 (m, 1H), 2.57 (s, 3H) and 2.49 (s, 3H).

### E 31 2-(4-Hydroxy-phenyl)-7-methyl-2′,3′-dihydro-[3,5′]bibenzofuranyl-5-ol

R⁶=1-Phenyl-vinyl

ES/MS m/z: 357 (neg. M−H); ¹H NMR (acetone-d6, 500 MHz): 7.55 (m, 2H), 7.29 (d, 1H, J=2.1 Hz), 7.18 (dd, 1H, J=8.2, 2.1 Hz), 7.00 (d, 1H, J=8.2 Hz), 6.83 (m, 2H), 6.71 (m, 1H), 6.66 (m, 1H), 3.72 (t, 2H, J=7.4 Hz), 3.22 (t, 2H, J=7.4 Hz) and 2.49 (s, 3H).

### E 32 2-(4-Hydroxy-phenyl)-7-methyl-3-(3-trifluoromethoxy-phenyl)-benzofuran-5-ol

R⁶=3-trifluoromethoxy-phenyl

ES/MS m/z: 399.0 (neg. M−H); ¹H NMR (acetone-d6, 500 MHz): 7.64 (m, 1H), 7.52-7.48 (m, 3H), 7.40-7.36 (m, 2H), 6.86 (m, 2H), 6.71 (m, 2H) and 2.50 (s, 3H).

### E 33 3-(2-Fluoro-pyridin-3-yl)-2-(4-hydroxy-phenyl)-7-methyl-benzofuran-5-ol

R⁶=2-fluoro-pyridin-3-yl

ES/MS m/z: 334.0 (neg. M−H); ¹H NMR (acetone-d6, 500 MHz): 8.32 (m, 1H), 8.03 (m, 1H), 7.48 (m, 1H), 7.46 (m, 2H), 6.87 (m, 2H), 6.71 (m, 1H), 6.57 (m, 1H) and 2.52 (s, 3H).

### E 34 3-Benzo[b]thiophen-2-yl-2-(4-hydroxy-phenyl)-7-methyl-benzofuran-5-ol

R⁶=benzo[b]thiophen-2-yl

ES/MS m/z: 373.3 (pos. M+H), 371.3 (neg. M−H).

### E 35 2-(4-Hydroxy-phenyl)-7-methyl-3-(1-methyl-1H-pyrazol-4-yl)-benzofuran-5-ol

R⁶=1-methyl-1H-pyrazol-4-yl

ES/MS ink: 321.3 (pos. M+H).

### E 36 2-(4-Hydroxy-phenyl)-7-methyl-3-(1-phenyl-vinyl)-benzofuran-5-ol

R⁶=1-phenyl-vinyl

ES/MS m/z: 343.3 (pos. M+H).

### E 37 2-(4-Hydroxy-phenyl)-7-methyl-3-pyridin-4-yl-benzofuran-5-ol

R⁶=pyridine-4-yl

ES/MS m/z: 318.3 (pos. M+H).

### E 38 2-(4-Hydroxy-phenyl)-7-methyl-3-(1-methyl-1H-pyrrol-2-yl)-benzofuran-5-ol

R⁶=1-methyl-1H-pyrrol-2-yl

ES/MS m/z: 320.4 (pos. M+H).

### E 39 3-(3,5-Dimethyl-isoxazol-4-yl)-2-(4-hydroxy-phenyl)-7-methyl-benzofuran-5-ol

R⁶=3,5-dimethyl-isoxazol-4-yl

ES/MS m/z: 336.3 (pos. M+H), 334.3 (neg. M−H); ¹H NMR (acetone-d6, 500 MHz): 7.53 (m, 2H), 6.91 (m, 2H), 6.70 (m, 1H), 6.52 (m, 1H), 2.51 (s, 3H), 2.24 (s, 3H) and 1.99 (s, 3H).

### E 40 3-(5-Fluoro-2-methyl-phenyl)-2-(4-hydroxy-phenyl)-7-methyl-benzofuran-5-ol

R⁶=5-fluoro-2-methyl-phenyl

ES/MS m/z: 349.3 (pos. M+H), 347.3 (neg. M−H); ¹H NMR (acetone-d6, 500 MHz): 7.44-7.39 (m, 3H), 7.15 (m, 1H), 7.08 (dd, 1H, J=9.5, 2.8 Hz), 6.82 (m, 2H), 6.69 (m, 1H), 6.40 (m, 1H), 2.52 (s, 3H) and 2.06 (s, 3H).

### E 41 3-[1-(4-Fluoro-phenyl)-vinyl]-2-(4-hydroxy-phenyl)-7-methyl-benzofuran-5-ol

R⁶=1-(4-fluoro-phenyl)-vinyl

ES/MS m/z: 361.4 (pos. M+H), 359.4 (neg. M−H); ¹H NMR (acetone-d6, 500 MHz): 7.66 (m, 2H), 7.51 (m, 2H), 7.06 (m, 2H), 6.82 (m, 2H), 6.66 (m, 1H), 6.48 (m, 1H), 6.01 (d, 1H, J=1.0 Hz), 5.45 (d, 1H; J=1.0 Hz), 2.50 (s, 3H).

### E 42 3-Cyclopropyl-2-(4-hydroxy-phenyl)-7-methyl-benzofuran-5-ol

R⁶=cyclopropyl

ES/MS m/z: 279.4 (neg. M−H).

### E 43 3-(5-Fluoro-2-methoxy-phenyl)-2-(4-hydroxy-phenyl)-7-methyl-benzofuran-5-ol

R⁶=5-fluoro-2-methoxy-phenyl

ES/MS m/z: 365.3 (pos. M+H), 363.3 (neg. M−H); ¹H NMR (acetone-d6, 500 MHz): 7.46 (m, 2H), 7.22-7.15 (m, 2H), 7.09 (dd, 1H, J=9.1, 2.9 Hz), 6.82 (m, 2H), 6.66 (m, 1H), 6.50 (m, 1H), 3.65 (s, 3H) and 2.50 (s, 3H).

### E 44 2-(4-Hydroxy-phenyl)-7-methyl-3-(1H-pyrrol-2-yl)-benzofuran-5-ol

R⁶=1H-pyrrol-2-yl

ES/MS m/z: 306.3 (pos. M+H), 304.3 (neg. M−H); ¹H NMR (acetone-d6, 500 MHz): 7.55 (m, 2H), 6.93 (m, 1H), 6.85 (m, 2H), 6.76 (m, 1H), 6.66 (m, 1H), 6.29-6.26 (m, 2H) and 2.48 (s, 3H).

### E 45 3-Furan-2-yl-2-(4-hydroxy-phenyl)-7-methyl-benzofuran-5-ol

R⁶=furan-2-yl

ES/MS m/z: 307.3 (pos. M+H), 305.3 (neg. M−H); ¹H NMR (acetone-d6, 500 MHz): 7.67 (t, 1H, J=1.3 Hz), 7.64 (m, 2H), 6.98 (d, 1H, J=1.3 Hz), 6.94 (m, 2H), 6.71 (m, 1H), 6.62-6.60 (m, 2H) and 2.48 (s, 3H).

### E 46 2-(4-Hydroxy-phenyl)-7-methyl-3-thiazol-5-yl-benzofuran-5-ol

R⁶=thiazol-5-yl

ES/MS m/z: 324.3 (pos. M+H), 322.3 (neg. M−H); ¹H NMR (acetone-d6, 500 MHz): 9.10 (s, 1H), 7.97 (s, 1H), 7.56 (m, 2H), 6.91 (m, 2H), 6.75-6.72 (m, 2H) and 2.50 (s, 3H).

### E 47 2-(4-Hydroxy-phenyl)-3-(2-methoxy-thiazol-4-yl)-7-methyl-benzofuran-5-ol

R⁶=2-methoxy-thiazol-4-yl

ES/MS m/z: 354.3 (pos. M+H), 352.2 (neg. M−H); ¹H NMR (acetone-d6, 500 MHz): 7.60 (m, 2H), 6.96 (m, 2H), 6.72 (m, 1H), 6.66 (m, 1H), 6.47 (s, 1H), 3.01 (s, 3H) and 2.51 (s, 3H).

### E 48 2-(4-Hydroxy-phenyl)-7-methyl-3-thiazol-2-yl-benzofuran-5-ol

R⁶=thiazol-2-yl

ES/MS m/z: 324.2 (pos. M+H), 322.2 (neg. M−H); ¹H NMR (acetone-d6, 500 MHz): very weak 9.10 (s, 1H), 7.97 (s, 1H), 7.57 (m, 2H), 6.91 (m, 2H), 6.75 (m, 1H), 6.73 (m, 1H) and 2.50 (s, 3H).

### E 49 2-(4-Hydroxy-phenyl)-3-(2-isopropyl-phenyl)-7-methyl-benzofuran-5-ol

R⁶=2-isopropyl-phenyl

ES/MS m/z: 359.3 (pos. M+H), 357.3 (neg. M−H); ¹H NMR (acetone-d6, 500 MHz): 7.54 (dd, 1H, J=7.8, 1.2 Hz), 7.47 (m, 1H), 7.41 (m, 2H), 7.32 (m, 1H), 7.23 (dd, 1H, J=7.5, 1.4 Hz), 6.78 (m, 2H), 6.67 (m, 1H), 6.36 (m, 1H), 2.93 (m, 1H), 2.52 (s, 3H), 1.10 (d, 3H, J=6.9 Hz) and 1.00 (d, 3H, J=6.9 Hz).

### E 50 3-(2-Ethyl-phenyl)-2-(4-hydroxy-phenyl)-7-methyl-benzofuran-5-ol

R⁶=2-ethyl-phenyl

ES/MS m/z: 345.3 (pos. M+H), 343.3 (neg. M−H); ¹H NMR (acetone-d6, 500 MHz): 7.46-7.40 (m, 4H), 7.33 (m, 1H), 7.26 (m, 1H), 6.78 (m, 2H), 6.66 (m, 1H), 6.35 (m, 1H), 2.52 (s, 3H), 2.48 (m, 21-1) and 0.96 (t, 3H, J=07.5 Hz).

### E 51 (E)-3-{2-[5-Hydroxy-2-(4-hydroxy-phenyl)-7-methyl-benzofuran-3-yl]-phenyl}-acrylonitrile

R⁶=2-((E)-2-Cyano-vinyl)-phenyl

ES/MS m/z: 368.3 (pos. M+H), 366.3 (neg. M−H); ¹H NMR (acetone-d6, 500 MHz): not pure 7.64-7.55 (m, 3H), 7.47 (dd, 1H, J=7.6, 1.4 Hz), 7.38-7.31 (m, 3H), 6.80 (m, 2H), 6.71 (m, 1H), 6.39 (m, 1H), 6.20 (d, 1H, J=16.6 Hz) and 2.53 (s, 3H).

### E 52 3-(2-Butoxy-phenyl)-2-(4-hydroxy-phenyl)-7-methyl-benzofuran-5-ol

R⁶=2-butoxy-phenyl

ES/MS m/z: 389.3 (pos. M+H), 387.3 (neg. M−H); ¹H NMR (acetone-d6, 500 MHz): 7.47 (m, 2H), 7.40 (m, 1H), 7.34 (dd, 1H, J=7.4, 1.7 Hz), 7.13 (d, 1H, J=8.0 Hz), 7.06 (m, 1H), 6.80 (m, 2H), 6.64 (m, 2H), 6.53 (m, 1H), 3.88 (m, 2H), 2.50 (s, 3H), 1.34 (m, 2H), 1.10 (m, 2H) and 0.71 (t, 3H, J=7.3 Hz).

### E 53 2-(4-Hydroxy-phenyl)-7-methyl-3-(2-trifluoromethoxy-phenyl)-benzofuran-5-ol

R⁶=2-trifluoromethoxy-phenyl

ES/MS m/z: 401.3 (pos. M+H), 399.3 (neg. M−H); ¹H NMR (acetone-d6, 500 MHz): 7.62 (m, 1H), 7.55-7.52 (m, 3H), 7.42 (m, 2H), 6.83 (m, 2H), 6.68 (m, 1H), 6.50 (m, 1H) and 2.51 (s, 3H).

### E 54 4-[5-Hydroxy-2-(4-hydroxy-phenyl)-7-methyl-benzofuran-3-yl]-thiophene-2-carbaldehyde

R⁶=thiophen-2-carbaldehyde

ES/MS m/z: 351.3 (pos. M+H), 349.2 (neg. M−H); ¹H NMR (acetone-d6, 500 MHz): 10.03 (s, 1H), 8.09 (s, 1H), 8.03 (s, 1H), 7.55 (m, 2H), 6.89 (m, 2H), 6.76 (m, 1H), 6.71 (m, 1H) and 2.50 (s, 3H).

### E 55 3-((E)-2-Cyclopropyl-vinyl)-2-(4-hydroxy-phenyl)-7-methyl-benzofuran-5-ol

R⁶=(E)-2-cyclopropyl-vinyl

ES/MS m/z: 307.3 (pos. M+H), 305.3 (neg. M−H); ¹H NMR (acetone-d6, 500 MHz): 7.67 (m, 2H), 7.02-6.99 (m, 3H), 6.73-6.65 (m, 2H), 5.85 (dd, 1H, J=16.0, 9.1 Hz), 2.53 (s, 3H), 1.66 (m, 1H), 0.85 (m, 2H) and 0.57 (m, 2H).

### E 56 2-(4-Hydroxy-phenyl)-7-methyl-3-(3-methyl-thiophen-2-yl)-benzofuran-5-ol

R⁶=3-methyl-thiophen-2-yl

ES/MS m/z: 410.3 (pos. M+H), 408.3 (neg. M−H); ¹H NMR (acetone-d6, 500 MHz): 7.54 (d, 1H, J=5.1 Hz), 7.52 (m, 2H), 7.08 (d, 1H, J=5.1 Hz), 6.85 (m, 2H), 6.68 (m, 1H), 6.59 (m, 1H), 2.51 (s, 3H) and 1.99 (s, 3H).

### E 57 2-[5-Hydroxy-2-(4-hydroxy-phenyl)-7-methyl-benzofuran-3-yl]-thiophene-3-carbaldehyde

R⁶=thiophene-2-yl-3-carbaldehyde

ES/MS m/z: 349.2 (pos. M+H), 347.2 (neg. M−H).

### E 58 2-[5-Hydroxy-2-(4-hydroxy-phenyl)-7-methyl-benzofuran-3-yl]-thiophene-3-carbonitrile

R⁶=3-cyano-thiophen-2-yl

ES/MS m/z: 410.3 (pos. M+H), 408.3 (neg. M−H); NMR (acetone-d6, 500 MHz): 7.54 (d, 1H, J=5.1 Hz), 7.52 (m, 2H), 7.08 (d, 1H, J=5.1 Hz), 6.85 (m, 2H), 6.68 (m, 1H), 6.59 (m, 1H), 2.51 (s, 3H) and 1.99 (s, 3H).

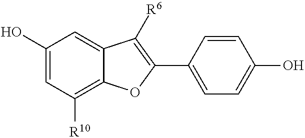

### E 59 7-Bromomethyl-2-(4-hydroxy-phenyl)-3-phenyl-benzofuran-5-ol

R⁶=phenyl R¹⁰=bromomethyl

ES/MS m/z: 395.0+397.1 (pos. M+H), 393.1+394.9 (neg. M−H); ¹H NMR MeOH-d4: 7.35-7.47 (m, 7H), 6.80 (d, 2.40 Hz, 1H), 6.70-6.74 (m, 3H), 4.79 (s, 2H).

### E 60 5-Hydroxy-2-(4-hydroxy-phenyl)-3-thiophen-3-yl-benzofuran-7-carbonitrile

R⁶=thiophen-3-yl R¹⁰=cyano

ES/MS m/z: 344.4 (pos. M+H), 393.1+332.2 (neg. M−H); ¹H NMR MeOH-d4: 7.59 (dd, 2.87 Hz, 5.02 Hz, 1H), 7.52 (dd, 1.30 Hz, 2.87 Hz, 1H), 7.48-7.52 (m, 2H), 7.10-7.14 (m, 2H), 7.03 (d, 2.51 Hz, 1H), 6.77-6.81 (m, 2H)

### E 61 5-Hydroxy-2-(4-hydroxy-phenyl)-3-phenyl-benzofuran-7-carbaldehyde

R⁶=phenyl, R¹⁰=carbaldehyde

ES/MS m/z: 331.3 (pos. M+H), 329.3 (neg. M−H); ¹H NMR MeOH-d4: 10.44 (s, 1H), 3.40-3.51 (m, 7H), 7.21 (d, 2.52 Hz, 1H), 7.06 (d, 2.53 Hz, 1H), 6.71-6.76 (m, 2H)

### E 62 7-Chloro-2-(4-hydroxy-phenyl)-3-phenyl-benzofuran-5-ol

R⁶=phenyl R¹⁰=chloro

ES/MS m/z: 337.2 (pos. M+H), 335.3 (neg. M−H); ¹H NMR MeOH-d4: 7.45-7.50 (m, 2H), 7.38-7.45 (m, 5H), 6.80 (d, 2.22 Hz, 1H), 6.71-6.75 (m, 2H), 6.69 (d, 2.22 Hz, 1H).

### E 63 7-Chloro-2-(4-hydroxy-phenyl)-3-thiophen-3-yl-benzofuran-5-ol

R⁶=thiophen-3-yl R¹⁰=chloro

ES/MS m/z: 343.2 (pos. M+H), 341.2 (neg. M−H); ¹H NMR MeOH-d4: 7.56 (dd, 2.85 Hz, 5.02 Hz, 1H), 7.46-7.50 (m, 3H), 7.11 (dd, 1.25 Hz, 5.00 Hz, 1H), 6.80 (d, 2.25 Hz, 1H), 6.75-6.79 (m, 3H).

### 64 5-Hydroxy-2-(4-hydroxy-phenyl)-3-thiophen-3-yl-benzofuran-7-carbaldehyde

R⁶=thiophen-3-yl, R¹⁰=carbaldehyde

ES/MS m/z: 337.2 (pos. M+H), 335.2 (neg. M−H); ¹H NMR MeOH-d4: 10.45 (s, 1H), 7.59 (dd, 2.97 Hz, 4.88 Hz, 1H), 7.52-7.57 (m, 3H), 7.22-7.24 (m, 1H), 7.14-7.16 (m, 2H), 6.86-6.81 (m, 2H)

### E 65 2-(4-Hydroxy-phenyl)-3-thiophen-3-yl-7-vinyl-benzofuran-5-ol

R⁶=thiophen-3-yl R¹⁰=vinyl

ES/MS m/z: 335.3 (pos. M+H), 333.2 (neg. M−H); ¹H NMR MeOH-d4: 7.56 (dd, 3.10 Hz, 5.06 Hz, 1H), 7.46-7.52 (m, 3H), 7.13 (dd, 1.24 Hz, 5.03 Hz, 1H), 6.96 (dd, 11.25 Hz, 17.77 Hz, 1H), 6.81 (d, 2.36 Hz, 1H), 6.74-6.80 (m, 3H), 6.23 (dd, 1.36 Hz, 11.32 Hz), 5.52 (dd, 1.36 Hz, 11.32 Hz, 1H)

### E 66 2-[7-Chloro-5-hydroxy-2-(4-hydroxy-phenyl)-benzofuran-3-yl]-thiophene-3-carbonitrile

R⁶=3-cyano-thiophen-2-yl R¹⁰=chloro

ES/MS m/z: 368.2 (pos. M+H), 366.2 (neg. M−H).

### E 67 3-(3-Cyano-furan-2-yl)-5-hydroxy-2-(4-hydroxy-phenyl)-benzofuran-7-carbonitrile

R⁶=3-cyano-furan-2yl R¹⁰=cyano

ES/MS m/z: 343.1 (pos. M+H), 341.1 (neg. M−H). ¹H NMR MeOH-d4: 7.87 (d, 1.93 Hz, 1H), 7.48-7.52 (m, 2H), 7.24 (d, 2.23 Hz, 1H), 7.14 (d, 2.22 Hz, 1H), 6.95 (d, 1.91 Hz, 1H), 6.86-6.90 (m, 2H).

### E 68 2-[5-Hydroxy-2-(4-hydroxy-phenyl)-7-trifluoromethyl-benzofuran-3-yl]-furan-3-carbonitrile

R⁶=3-cyano-furan-2yl R¹⁰=trifluoromethyl

ES/MS m/z: 386.1 (pos. M+H), 384.0 (neg. M−H). ¹H NMR MeOH-d4: 7.86 (d, 2.21 Hz, 1H), 7.44-7.49 (m, 2H), 7.15 (d, 2.24 Hz, 1H), 7.08 (d, 2.30 Hz, 1H), 6.83-6.90 (m, 3H).

### E 69 2-[2-(3-Fluoro-4-hydroxy-phenyl)-5-hydroxy-7-methyl-benzofuran-3-yl]-furan-3-carbonitrile

R⁶=3-cyano-furan-2yl R=3-fluoro-4-hydroxy-phenyl

ES/MS m/z: 350.1 (pos. M+H), 348.1 (neg. M−H). ¹H NMR MeOH-d4: 7.84 (d, 2.19 Hz, 1H), 7.30 (dd, 2.05 Hz, 12.06 Hz, 1H), 7.22-7.26 (m, 1H), 6.97 (t, 8.78 Hz, 1H), 6.92 (d, 2.21 Hz, 1H), 6.77 (d, 2.21 Hz, 1H), 6.68-6.71 (m, 1H), 2.51 (s, 3H).

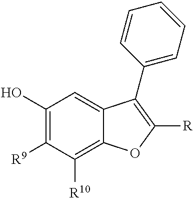

### E 70 2-(4-Hydroxy-phenyl)-6-methyl-3-phenyl-benzofuran-5-ol

R=4-hydroxyphenyl R⁹=methyl

R¹⁰=hydrogen

ES/MS m/z: 317.0 (pos. M+H), 315.1 (neg. M−H). ¹H NMR MeOH-d4: 7.34-7.48 (m, 7H), 7.22 (s, 1H), 6.76 (s, 1H), 6.68-6.72 (m, 2H), 2.30 (s, 1H).

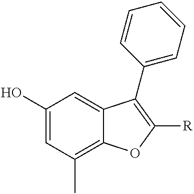

### E 71 2-(2,5-Difluoro-4-hydroxy-phenyl)-7-methyl-3-phenyl-benzofuran-5-ol

R=2,5-difluoro-4-hydroxy-phenyl

ES/MS m/z: 335.6 (pos. M+H), 333.4 (neg. M−H); ¹H NMR MeOH-d4: 7.34-7.42 (m, 4H), 7.29-7.34 (m, 1H), 7.15 (dd, 6.61 Hz, 11.34 Hz, 1H), 6.79 (d, 2.21 Hz, 1H), 6.63-6.69 (m, 2H), 2.48 (s, 3H)

### E 72 2-(2,6-Difluoro-4-hydroxy-phenyl)-7-methyl-3-phenyl-benzofuran-5-ol

R=2,6-difluoro-4-hydroxy-phenyl

ES/MS m/z: 335.6 (pos. M+H), 333.1 (neg. M−H); ¹H NMR MeOH-d4: 7.33-7.38 (m, 4H), 7.26-7.30 (m, 1H), 6.87 (d, 2.45 Hz, 1H), 6.69 (d, 2.45 Hz, 1H), 6.42 (d, 9.37 Hz, 2H), 2.47 (s, 3H)

### E 73 2-(3-Fluoro-4-hydroxy-phenyl)-7-methyl-3-phenyl-benzofuran-5-ol

R=3-fluoro-4-hydroxy-phenyl

ES/MS m/z: 353.3 (pos. M+H), 351.4 (neg. M−H); NMR MeOH-d4: 7.46-7.51 (m, 2H), 7.39-7.44 (m, 3H), 7.20-7.26 (m, 2H), 6.81-6.86 (m, 1H), 6.63 (d, 2.27 Hz, 1H), 6.58 (d, 2.27 Hz, 1H), 2.50 (s, 3H)

### E 74 2-(1H-Indazol-5-yl)-7-methyl-3-phenyl-benzofuran-5-ol

R=1H-indazol-5-yl

ES/MS m/z: 341.3 (pos. M+H), 339.1 (neg. M−H); ¹H NMR MeOH-d4: 8.04-8.06 (m, 1H), 7.97-8.01 (m, 1H), 7.56-7.59 (m, 1H), 7.35-7.47 (m, 6H), 6.68 (d, 2.18 Hz, 1H), 6.63-6.66 (m, 1H), 2.54 (s, 3H).

### 75 2-(3,5-Difluoro-4-hydroxy-phenyl)-7-methyl-3-phenyl-benzofuran-5-ol

R=3,5-difluoro-4-hydroxy-phenyl

ES/MS m/z: 353.2 (pos. M+H), 351.3 (neg. M−H); ¹H NMR MeOH-d4: 7.49-7.54 (m, 2H), 7.42-7.47 (m, 3H), 7.07-7.11 (m, 2H), 6.65 (d, 2.28 Hz, 1H), 6.56 (d, 2.28 Hz, 1H)

### E 76 2-(3-Chloro-5-fluoro-4-hydroxy-phenyl)-7-methyl-3-phenyl-benzofuran-5-ol

R=3-chloro-5-fluoro-4-hydroxy-phenyl

ES/MS m/z: 368.9 (pos. M+H), 367.0 (neg. M−H); ¹H NMR MeOH-d4: 7.48-7.54 (m, 2H), 4.41-7.47 (m, 3H), 7.33-7.35 (m, 1H), 7.17 (dd, 2.21 Hz, 11.79 Hz, 1H), 6.64-6.66 (m, 1H), 7.57 (d, 2.30 Hz, 1H), 2.51 (s, 3H)

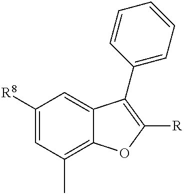

### E 77 5-(5-Methoxy-7-methyl-3-phenyl-benzofuran-2-yl)-1H-indazole

R=1H-indazole R⁸=methoxy

ES/MS m/z: 355.4 (pos. M+H), 353.2 (neg. M−H). ¹H NMR CDCl₃: 8.10-8.13 (m, 2H), 7.66 (d, 8.73 Hz, 1H), 7.40-7.53 (m, 6H), 6.76-6.79 (m, 2H), 3.80 (s, 3H), 2.60 (s, 3H)

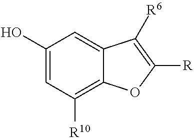

### E 78 2-(3-Fluoro-4-hydroxy-phenyl)-7-methyl-3-thiophen-3-yl-benzofuran-5-ol

R⁶=thiophen-3-yl R=3-fluoro-4-hydroxy-phenyl

R¹⁰=methyl

ES/MS m/z: 341.3 (pos. M+H), 339.1 (neg. M−H); ¹H NMR MeOH-d4: 7.58 (dd, 3.16 Hz, 4.73 Hz, 1H), 7.48 (dd, 1.28 Hz, 3.16 Hz, 1H), 7.27-7.31 (m, 2H), 7.13 (dd, 1.25 Hz, 4.73 Hz, 1H), 6.85-6.90 (m, 1H), 6.64-6.66 (m, 1H), 6.62-6.63 (m, 1H), 2.50 (s, 3H)

### E 79 2-(2-Fluoro-4-hydroxy-phenyl)-7-methyl-3-thiophen-3-yl-benzofuran-5-ol

R⁶=thiophen-3-yl R=2-fluoro-4-hydroxy-phenyl

R¹⁰=methyl

ES/MS m/z: 341.3 (pos. M+H), 339.1 (neg. M−H); ¹H NMR MeOH-d4: 7.42 (dd, 2.83 Hz, 5.04 Hz, 1H), 7.38 (dd, 1.25 Hz, 2.83 Hz, 1H), 7.30 (t, 8.49 Hz, 1H), 7.03 (dd, 1.26 Hz, 5.04 Hz, 1H), 6.88-6.89 (m, 1H), 6.63-6.66 (m, 2H), 6.57 (dd, 2.22 Hz, 11.67 Hz, 1H), 2.47 (s, 3H)

### E 80 2-(2,6-Difluoro-4-hydroxy-phenyl)-7-methyl-3-thiophen-3-yl-benzofuran-5-ol

R⁶=thiophen-3-yl R=2,6-difluoro-4-hydroxy-phenyl

R¹⁰=methyl

ES/MS m/z: 359.3 (pos. M+H), 357.1 (neg. M−H); ¹H NMR MeOH-d4: 7.42 (dd, 2.83 Hz, 5.00 Hz, 1H), 7.38 (dd, 1.28 Hz, 2.83 Hz, 1H), 7.05 (dd, 1.28 Hz, 5.00 Hz, 1H), 6.96 (d, 2.50 Hz, 1H), 6.69 (d, 2.50 Hz, 1H), 6.47 (d, 9.27 Hz, 1H), 2.46 (s, 3H)

### E 81 2-(3-Chloro-5-fluoro-4-hydroxy-phenyl)-7-methyl-3-thiophen-3-yl-benzofuran-5-ol

R⁶=thiophen-3-yl R=3-chloro-5-fluoro-4-hydroxy-phenyl

R¹⁰=methyl

ES/MS m/z: 373.1 (neg. M−H); ¹H NMR MeOH-d4: 7.62 (dd, 2.84 Hz, 5.04 Hz, 1H), 7.50 (dd, 1.27 Hz, 2.84 Hz, 1H), 7.40 (t, 1.87 Hz, 1H), 7.24 (1.93 Hz, 5.04 Hz), 7.14 (dd, 1.27 Hz, 5.04 Hz, 1H), 6.63-6.66 (m, 2H), 2.50 (s, 3H)

### E 82 2-[2-(3-Chloro-5-fluoro-4-hydroxy-phenyl)-5-hydroxy-7-methyl-benzofuran-3-yl]-furan-3-carbonitrile

R⁶=3-cyano-furan-2yl R=3-chloro-5-fluoro-4-hydroxy-phenyl

R¹⁰=methyl

ES/MS m/z: 384.0 (pos. M+H), 382.0 (neg. M−H). ¹H NMR MeOH-d4: 7.87 (d, 1.97 Hz, 1H), 7.35-7.38 (m, 1H), 7.26 (dd, 2.14 Hz, 11.36 Hz, 1H), 6.94 (d, 1.95 Hz, 1H), 6.79 (d, 2.22 Hz, 1H), 6.72 (d, 2.16 Hz, 1H), 2.52 (s, 3H)

### E 83 2-[7-Fluoro-5-hydroxy-2-(1H-indazol-5-yl)-benzofuran-3-yl]-furan-3-carbonitrile

R⁶=3-cyano-furan-2yl R=1H-indazol-5-yl

R¹⁰=fluoro

ES/MS m/z: 360.0 (pos. M+H), 358.1 (neg. M−H). ¹H NMR MeOH-d4: 8.12-8.15 (m, 2H), 7.87 (d, 2.19 Hz, 1H), 7.61-7.64 (m, 1H), 7.58 (dd, 1.55 Hz, 8.11 Hz, 1H), 6.94 (d, 2.19 Hz, 1H), 6.81 (d, 2.19 Hz, 1H), 6.70 (dd, 2.22 Hz, 12.30 Hz, 1H)

### Example 84

### Description of the Estrogen Receptor Binding Assays

The estrogen receptor ligand binding assays are designed as scintillation proximity assays (SPA), employing the use of tritiated estradiol (³H-E2) and recombinant expressed biotinylated estrogen receptor binding domains. The binding domains of human ERα (ERα-LBD, pET-N-AT #1, aa 301-595) and ERβ (ERβ-LBD, pET-N-AT #1, aa 255-530) proteins are produced in E. coli ((BL21, (DE3), pBirA)) at 22 C in 2×LB medium supplemented with 50 uM biotin. After 3 h of IPTG induction (0.55 mM), cells are harvested by centrifugation at 7300×g for 15 min and cell pellets stored frozen in −20 C. Extraction of ERα and ERβ are performed using 5 g of cells suspended in 50 mL of extraction buffer (50 mM Tris, pH 8.0, 100 mM KCl, 4 mM EDTA, 4 mM DDT and 0.1 mM PMSF). The cell suspension is run twice through a Microfluidizer M-110L (Microfluidics) and centrifuged at 15,000×g for 60 min. The supernatant is aliquoted and stored in −70 C.

Dilute ERα-LBD or ERβ-LBD extracts in assay buffer (18 mM K₂HPO₄, 2 mM KH₂PO₄, 20 mM Na₅MoO₄, 1 mM EDTA, 1 mM TCEP) 1:676 and 1:517 for alpha and beta respectively. The diluted receptor concentrations should be 900 fmol/L. Preincubate the extracts with streptavidin coated polyvinyltoluene SPA beads (RPNQ0007, GE Healthcare) at a concentration of 0.43 mg/mL for 1 hr at room temperature.

Test compounds are evaluated over a range of concentrations from 157 μM to 37.5 μM. The test compound stock solutions should be made in 100% DMSO at 5× of the final concentration desired for testing in the assay. The amount of DMSO in the test wells of the 384 well plate will be 20%. Add 18 μl aliquots of test compounds to the assay plates followed by 35 μl of the preincubated receptor/SPA bead mix and finally add 350 of 3 nM ³H-E2. Cover the plates with a plastic sealer, centrifuge for 1 minute at 1000 rpm and equilibrate over night on a shaker at room temperature. The following morning, centrifuge the plates 5 minutes at 2000 rpm and measure on a plate scintillation counter e.g. a PerkinElmer Microbeta 1450 Trilux.

For compounds able to displace 3 [H]-E2 from the receptor an IC₅₀-value (the concentration required to inhibit 50% of the binding of 3 [H]-E2) is determined by a non-linear four parameter logistic model; b=((bmax−bmin)/(1+(I/IC₅₀)S))+bmin I is added concentration of binding inhibitor, IC₅₀ is the concentration of inhibitor at half maximal binding and S is a slope factor. The Microbeta-instrument generates the mean cpm (counts per minute) value/minute and corrects for individual variations between the detectors thus generating corrected cpm values.

The compounds of Examples 1-84 exhibit binding affinities to the estrogen receptor α-subtype in the range of IC₅₀ 1 to 10,000 nM or to the estrogen receptor β-subtype in the range of IC₅₀ 1 to 10,000 nM.

1-26. (canceled)

27. A compound of formula (I) or a pharmaceutically acceptable ester, amide, solvate or salt thereof, including a salt of such an ester or amide, and a solvate of such an ester, amide or salt: 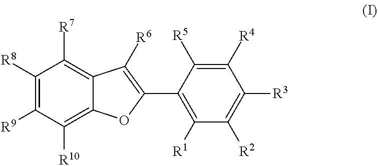
wherein R¹, R², R⁴ and a R⁵ are each independently selected from the group consisting of hydrogen, OR^{A}, halogen, cyano, nitro, C₁₋₆alkyl, C₂₋₆alkenyl, C₂₋₆alkynyl, dihaloC₁₋₆alkyl and trihaloC₁₋₆alkyl;
R³ is selected from the group consisting of OR^{A}; —CHO; —C(O)C₁₋₄alkyl; —C(O)phenyl; —O—C(O)R^{A}; and N(R^{B})₂ in which each R^{B} is independently selected from the group consisting of hydrogen, —C(O)C₁₋₄alkyl, —C(O)phenyl, —SO₂C₁₋₄alkyl, —SO₂phenyl, C₁₋₆alkyl, C₂₋₆alkenyl, C₂₋₆alkynyl, C₃₋₈cycloalkyl, C₃₋₈cycloalkyl-C₁₋₆alkyl, C₆₋₁₀aryl-C₁₋₆alkyl, C₅₋₁₀heterocyclyl and C₅₋₁₀heterocyclyl-C₁₋₆ alkyl, with the proviso that R³ is not —NH₂;
or R³ and R⁴ together with the atoms to which they are attached, form a 5-, 6- or 7-membered cyclic group optionally containing one to three heteroatoms selected from O, N and S, said 5-, 6- or 7-membered cyclic group being optionally substituted with 1 or 2 groups selected from OR^{A}, halogen, cyano, nitro, C₁₋₆alkyl, C₂₋₆alkenyl, C₂₋₆alkynyl, dihaloC₁₋₆alkyl and trihaloC₁₋₆alkyl;
R⁶ is selected from the group consisting of C₃₋₈cycloalkyl, C₃₋₈cycloalkyl-C₁₋₆alkyl, C₃₋₈cycloalkyl-C₁₋₆alkenyl, phenyl, biphenyl, phenyl-C(═CH₂)—, and C₅₋₁₀heterocyclyl, wherein said phenyl, biphenyl, phenyl-C(═CH₂)— or C₅₋₁₀heterocyclyl group is unsubstituted or substituted on the ring with 1 to 3 substituents, each substituent being selected from the group consisting of OR^{A}; halogen; cyano; nitro; —CHO; —CO.C₁₋₆alkyl; C₁₋₆alkyl, C₁₋₆alkoxy or C₁₋₆alkoxyalkyl optionally substituted by 1 to 3 halogen atoms; C₂₋₆alkenyl optionally substituted by halogen or cyano; C₂₋₆alkynyl; SO₂H; SO₂C₁₋₆alkyl; SH; and SC₁₋₆alkyl;
R⁸ is OR^{A};
R⁷, R⁹ and R¹⁰ are each independently selected from the group consisting of hydrogen, OR^{A}, halogen, cyano, nitro, C₁₋₆alkyl, C₂₋₆alkenyl, C₂₋₆alkynyl, C(O)H, C(O)C₁₋₆alkyl, haloC₁₋₆alkyl, dihaloC₁₋₆alkyl, trihaloC₁₋₆alkyl, cyanoC₁₋₆alkyl, and C₁₋₄alkoxyC₁₋₆alkyl; and
R^{A} is selected from the group consisting of hydrogen, C₁₋₆alkyl, C₂₋₆alkenyl, C₂₋₆alkynyl, C₃₋₈cycloalkyl, C₃₋₈cycloalkyl-C₁₋₆alkyl, C₆₋₁₀aryl and C₆₋₁₀ aryl-C₁₋₆alkyl;
with the provisos that (i) if all of R², R³, R⁸ and R⁹ represent methoxy groups and all of R¹, R⁴, R⁵, R⁷ and R¹⁰ represent hydrogen atoms, then R⁶ represents a group other than phenyl or 3,4-dimethoxyphenyl; (ii) if both R³ and R⁸ represent methoxy groups, R² represents an isopropoxy group, R⁹ represents a hydroxy or an isopropoxy group and all of R¹, R⁴, R⁵, R⁷ and R¹⁰ represent hydrogen atoms, then R⁶ represents a group other than 3,4,5-trimethoxyphenyl; (iii) if both of R³ and R⁸ represent methoxy groups and all of R¹, R², R⁴, R⁵, R⁷, R⁹ and R¹⁰ represent hydrogen atoms, then R⁶ represents a group other than phenyl; and (iv) the compound of formula (I) is not 2-(3′-hydroxy-4′-methoxyphenyl)-5-methoxy-3-(3″,4″,5″-trimethoxyphenyl)benzofuran-6-ol.

28. The compound of claim 27, in which R¹, R², and R⁵ are each independently selected from the group consisting of hydrogen, OR^{A}, halogen, cyano, nitro, C₁₋₄alkyl, haloC₁₋₄alkyl, dihaloC₁₋₄alkyl and trihaloC₁₋₄alkyl.

29. The compound of claim 28, in which R¹, R² and R⁵ are each independently selected from the group consisting of hydrogen, hydroxy, halogen, cyano, methyl and trifluoromethyl.

30. The compound of claim 27, in which R³ is selected from the group consisting of OR^{A} and N(R^{B})₂, in which each R^{B} is independently selected from the group consisting of hydrogen, C₁₋₄alkyl, and C₃₋₆cycloalkyl.

31. The compound of claim 30, in which R³ represents a hydroxy group.

32. The compound of claim 27, in which R⁴ represents hydrogen, OR^{A}, halogen, cyano, nitro, C₁₋₄alkyl, haloC₁₋₄alkyl, dihaloC₁₋₄alkyl or trihaloC₁₋₄alkyl.

33. The compound of claim 32, in which R⁴ represents hydrogen, hydroxy, halogen, cyano, methyl or trifluoromethyl.

34. The compound of claim 27, in which R³ and R⁴ together represent an —NH—CH═N—, —NH—N═CH— or —CH—CH—NH— group.

35. The compound of claim 27, in which R⁶ is selected from the group consisting of C₃₋₆cycloalkyl, C₃₋₆cycloalkyl-C₁₋₂alkyl, C₃₋₆cycloalkyl-C₁₋₂alkenyl, phenyl, biphenyl, phenyl-C(═CH₂)—, and C₅₋₇heterocyclyl wherein said phenyl, biphenyl, phenyl-C(═CH₂)— or C₅₋₁₀heterocyclyl group is unsubstituted or substituted on the ring with 1 or 2 substituents, each substituent being selected from the group consisting of OR^{A}; halogen; cyano; and C₁₋₄alkyl or C₁₋₄alkoxy optionally substituted by 1 to 3 halogen atoms.

36. The compound of claim 35, in which R⁶ is an aromatic group.

37. The compound of claim 35, in which R⁶ is an optionally substituted phenyl or C₅heterocyclyl group.

38. The compound of claim 27, in which R⁷, R⁹ and R¹⁰ are each independently selected from the group consisting of hydrogen, OR^{A}, halogen, cyano, C₁₋₄alkyl, haloC₁₋₄alkyl, dihaloC₄alkyl and trihaloC₁₋₄alkyl.

39. The compound of claim 38, in which R⁷, R⁹ and R¹⁰ are each independently selected from hydrogen, hydroxy, halogen, cyano, methyl and trifluoromethyl.

40. The compound of claim 27, in which R^{A} is selected from the group consisting of hydrogen, C₁₋₄alkyl, C₃₋₆cycloalkyl, C₃₋₆cycloalkyl-C₁₋₄alkyl, phenyl and benzyl.

41. The compound of claim 40, in which R^{A} represents hydrogen or C₁₋₄alkyl.

42. The compound of claim 27, in which:
R^{A} represents C₁₋₄alkyl or hydrogen;
R¹, R², and R⁵ are each independently selected from the group consisting of hydrogen, OR^{A}, halogen, cyano, halomethyl, dihalomethyl and trihalomethyl;
R³ represents OR^{A} or N(R^{B})₂, in which each R^{B} independently represents hydrogen or C₁₋₄alkyl;
R⁴ represents one of the preferred groups mentioned above for R¹, R², and R⁵; or
R³ and R⁴ together represent an —NH—N═CH— group;
R⁶ represents phenyl or a C₅heterocyclyl group which can either be unsubstituted or substituted on the ring with 1 or 2 substituents, each substituent being selected from the group consisting of OR^{A}; halogen; cyano; and C₁₋₄alkyl or C₁₋₄alkoxy optionally substituted by 1 to 3 halogen atoms; and
R⁷, R⁹ and R¹³ each independently represent hydrogen, OR^{A}, halogen, cyano, halomethyl, dihalomethyl and trihalomethyl.

43. The compound of claim 42, in which at least one of R¹, R², and R⁵ represents hydrogen; R³ represents OR^{A}; or R³ and R⁴ together represent an —NH—CH═N—, —NH—N═CH— or —CH═CH—NH— group; R⁶ represents phenyl or a C₅heterocyclyl group optionally substituted by cyano; and R⁷, R⁹ and R¹⁰ each independently represent hydrogen, hydroxy, halogen, cyano, methyl and trifluoromethyl, at least one of R⁷, R⁹ and R¹⁰ being hydrogen.

44. The compound of claim 27, which is selected from:
2-(4-Hydroxy-phenyl)-7-methyl-3-phenyl-benzofuran-5-ol;
5-Hydroxy-2-(4-hydroxy-phenyl)-3-phenyl-benzofuran-7-carbonitrile;
2-(2-Fluoro-4-hydroxy-phenyl)-7-methyl-3-phenyl-benzofuran-5-ol;
7-Dibromomethyl-2-(4-hydroxy-phenyl)-3-phenyl-benzofuran-5-ol;
[5-Hydroxy-2-(4-hydroxy-phenyl)-3-phenyl-benzofuran-7-yl]-acetonitrile;
2-(4-Hydroxy-phenyl)-7-(1-methoxy-ethyl)-3-phenyl-benzofuran-5-ol;
7-Difluoromethyl-2-(4-hydroxy-phenyl)-3-phenyl-benzofuran-5-ol;
2-(4-Hydroxy-phenyl)-3-phenyl-7-vinyl-benzofuran-5-ol;
2-(4-Hydroxy-phenyl)-3-thiophen-3-yl-7-trifluoromethyl-benzofuran-5-ol;
7-Fluoro-2-(1H-indazol-5-yl)-3-thiophen-3-yl-benzofuran-5-ol;
2-[7-Chloro-5-hydroxy-2-(4-hydroxy-phenyl)-benzofuran-3-yl]-furan-3-carbonitrile;
2-(4-Hydroxy-phenyl)-7-methyl-3-thiophen-3-yl-benzofuran-5-ol;
3-(2,5-Difluoro-phenyl)-2-(4-hydroxy-phenyl)-7-methyl-benzofuran-5-ol;
3-[5-Hydroxy-2-(4-hydroxy-phenyl)-7-methyl-benzofuran-3-yl]-benzonitrile;
2-(4-Hydroxy-phenyl)-7-methyl-3-m-tolyl-benzofuran-5-ol;
2-(4-Hydroxy-phenyl)-7-methyl-3-thiophen-2-yl-benzofuran-5-ol;
2-(4-Hydroxy-phenyl)-7-methyl-3-pyridin-3-yl-benzofuran-5-ol;
2-[5-Hydroxy-2-(4-hydroxy-phenyl)-7-methyl-benzofuran-3-yl]-benzonitrile;
2-(4-Hydroxy-phenyl)-7-methyl-3-(3-nitro-phenyl)-benzofuran-5-ol;
5-[5-Hydroxy-2-(4-hydroxy-phenyl)-7-methyl-benzofuran-3-yl]-thiophene-2-carbaldehyde;
3-(3,5-Difluoro-phenyl)-2-(4-hydroxy-phenyl)-7-methyl-benzofuran-5-ol;
3-(3,5-Dichloro-phenyl)-2-(4-hydroxy-phenyl)-7-methyl-benzofuran-5-ol;
2-(4-Hydroxy-phenyl)-7-methyl-3-(2-phenoxy-phenyl)-benzofuran-5-ol;
3-Biphenyl-2-yl-2-(4-hydroxy-phenyl)-7-methyl-benzofuran-5-ol;
3-(2-Hydroxy-phenyl)-2-(4-hydroxy-phenyl)-7-methyl-benzofuran-5-ol;
1-{3-[5-Hydroxy-2-(4-hydroxy-phenyl)-7-methyl-benzofuran-3-yl]-phenyl}-ethanone;
2-(4-Hydroxy-phenyl)-3-(3-methanesulfonyl-phenyl)-7-methyl-benzofuran-5-ol;
3-(3-Ethylsulfanyl-phenyl)-2-(4-hydroxy-phenyl)-7-methyl-benzofuran-5-ol;
2-(4-Hydroxy-phenyl)-7-methyl-3-quinolin-5-yl-benzofuran-5-ol;
1-{5-[5-Hydroxy-2-(4-hydroxy-phenyl)-7-methyl-benzofuran-3-yl]-thiophen-2-yl}-ethanone;
2-(4-Hydroxy-phenyl)-7-methyl-2′,3′-dihydro-[3,5′]bibenzofuranyl-5-ol;
2-(4-Hydroxy-phenyl)-7-methyl-3-(3-trifluoromethoxy-phenyl)-benzofuran-5-ol;
3-(2-Fluoro-pyridin-3-yl)-2-(4-hydroxy-phenyl)-7-methyl-benzofuran-5-ol;
3-Benzo[b]thiophen-2-yl-2-(4-hydroxy-phenyl)-7-methyl-benzofuran-5-ol;
2-(4-Hydroxy-phenyl)-7-methyl-3-(1-methyl-1H-pyrazol-4-yl)-benzofuran-5-ol;
2-(4-Hydroxy-phenyl)-7-methyl-3-(1-phenyl-vinyl)-benzofuran-5-ol;
2-(4-Hydroxy-phenyl)-7-methyl-3-pyridin-4-yl-benzofuran-5-ol;
2-(4-Hydroxy-phenyl)-7-methyl-3-(1-methyl-1H-pyrrol-2-yl)-benzofuran-5-ol;
3-(3,5-Dimethyl-isoxazol-4-yl)-2-(4-hydroxy-phenyl)-7-methyl-benzofuran-5-ol;
3-(5-Fluoro-2-methyl-phenyl)-2-(4-hydroxy-phenyl)-7-methyl-benzofuran-5-ol;
3-[1-(4-Fluoro-phenyl)-vinyl]-2-(4-hydroxy-phenyl)-7-methyl-benzofuran-5-ol;
3-Cyclopropyl-2-(4-hydroxy-phenyl)-7-methyl-benzofuran-5-ol;
3-(5-Fluoro-2-methoxy-phenyl)-2-(4-hydroxy-phenyl)-7-methyl-benzofuran-5-ol;
2-(4-Hydroxy-phenyl)-7-methyl-3-(1H-pyrrol-2-yl)-benzofuran-5-ol;
3-Furan-2-yl-2-(4-hydroxy-phenyl)-7-methyl-benzofuran-5-ol;
2-(4-Hydroxy-phenyl)-7-methyl-3-thiazol-5-yl-benzofuran-5-ol;
2-(4-Hydroxy-phenyl)-3-(2-methoxy-thiazol-4-yl)-7-methyl-benzofuran-5-ol;
2-(4-Hydroxy-phenyl)-7-methyl-3-thiazol-2-yl-benzofuran-5-ol;
2-(4-Hydroxy-phenyl)-3-(2-isopropyl-phenyl)-7-methyl-benzofuran-5-ol;
3-(2-Ethyl-phenyl)-2-(4-hydroxy-phenyl)-7-methyl-benzofuran-5-ol;
(E)-3-{2-[5-Hydroxy-2-(4-hydroxy-phenyl)-7-methyl-benzofuran-3-yl]-phenyl}-acrylonitrile;
3-(2-Butoxy-phenyl)-2-(4-hydroxy-phenyl)-7-methyl-benzofuran-5-ol;
2-(4-Hydroxy-phenyl)-7-methyl-3-(2-trifluoromethoxy-phenyl)-benzofuran-5-ol;
4-[5-Hydroxy-2-(4-hydroxy-phenyl)-7-methyl-benzofuran-3-yl]-thiophene-2-carbaldehyde;
3-((E)-2-Cyclopropyl-vinyl)-2-(4-hydroxy-phenyl)-7-methyl-benzofuran-5-ol;
2-(4-Hydroxy-phenyl)-7-methyl-3-(3-methyl-thiophen-2-yl)-benzofuran-5-ol;
2-[5-Hydroxy-2-(4-hydroxy-phenyl)-7-methyl-benzofuran-3-yl]-thiophene-3-carbaldehyde;
2-[5-Hydroxy-2-(4-hydroxy-phenyl)-7-methyl-benzofuran-3-yl]-thiophene-3-carbonitrile;
7-Bromomethyl-2-(4-hydroxy-phenyl)-3-phenyl-benzofuran-5-ol;
5-Hydroxy-2-(4-hydroxy-phenyl)-3-thiophen-3-yl-benzofuran-7-carbonitrile;
5-Hydroxy-2-(4-hydroxy-phenyl)-3-phenyl-benzofuran-7-carbaldehyde;
7-Chloro-2-(4-hydroxy-phenyl)-3-phenyl-benzofuran-5-ol;
7-Chloro-2-(4-hydroxy-phenyl)-3-thiophen-3-yl-benzofuran-5-ol;
5-Hydroxy-2-(4-hydroxy-phenyl)-3-thiophen-3-yl-benzofuran-7-carbaldehyde;
2-(4-Hydroxy-phenyl)-3-thiophen-3-yl-7-vinyl-benzofuran-5-ol;
2-[7-Chloro-5-hydroxy-2-(4-hydroxy-phenyl)-benzofuran-3-yl]-thiophene-3-carbonitrile;
3-(3-Cyano-furan-2-yl)-5-hydroxy-2-(4-hydroxy-phenyl)-benzofuran-7-carbonitrile;
2-[5-Hydroxy-2-(4-hydroxy-phenyl)-7-trifluoromethyl-benzofuran-3-yl]-furan-3-carbonitrile;
2-[2-(3-Fluoro-4-hydroxy-phenyl)-5-hydroxy-7-methyl-benzofuran-3-yl]-furan-3-carbonitrile;
2-(4-Hydroxy-phenyl)-6-methyl-3-phenyl-benzofuran-5-ol;
2-(2,5-Difluoro-4-hydroxy-phenyl)-7-methyl-3-phenyl-benzofuran-5-ol;
2-(2,6-Difluoro-4-hydroxy-phenyl)-7-methyl-3-phenyl-benzofuran-5-ol;
2-(3-Fluoro-4-hydroxy-phenyl)-7-methyl-3-phenyl-benzofuran-5-ol;
2-(1H-Indazol-5-yl)-7-methyl-3-phenyl-benzofuran-5-ol;
2-(3,5-Difluoro-4-hydroxy-phenyl)-7-methyl-3-phenyl-benzofuran-5-ol;
2-(3-Chloro-5-fluoro-4-hydroxy-phenyl)-7-methyl-3-phenyl-benzofuran-5-ol;
5-(5-Methoxy-7-methyl-3-phenyl-benzofuran-2-yl)-1H-indazole;
2-(3-Fluoro-4-hydroxy-phenyl)-7-methyl-3-thiophen-3-yl-benzofuran-5-ol;
2-(2-Fluoro-4-hydroxy-phenyl)-7-methyl-3-thiophen-3-yl-benzofuran-5-ol;
2-(2,6-Difluoro-4-hydroxy-phenyl)-7-methyl-3-thiophen-3-yl-benzofuran-5-ol;
2-(3-Chloro-5-fluoro-4-hydroxy-phenyl)-7-methyl-3-thiophen-3-yl-benzofuran-5-ol;
2-[2-(3-Chloro-5-fluoro-4-hydroxy-phenyl)-5-hydroxy-7-methyl-benzofuran-3-yl]-furan-3-carbonitrile; or
2-[7-Fluoro-5-hydroxy-2-(1H-indazol-5-yl)-benzofuran-3-yl]-furan-3-carbonitrile;
or a pharmaceutically acceptable ester, amide, solvate or salt thereof, including a salt of such an ester or amide, and a solvate of such an ester, amide or salt.

45. A method for the treatment or prophylaxis of a disease or disorder associated with estrogen receptor activity in a mammal, which comprises administering to the mammal a therapeutically effective amount of the compound of claim 27.

46. The method of claim 45, wherein the condition associated with a disease or disorder associated with estrogen receptor activity is selected from bone loss, bone fractures, osteoporosis, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression, autoimmune disease, inflammation, IBD, IBS, sexual dysfunction, hypertension, retinal degeneration and lung, colon, breast, uterus, and prostate cancer.

47. A pharmaceutical composition which comprises a compound of formula (I) or a pharmaceutically acceptable ester, amide, solvate or salt thereof, including a salt of such an ester or amide, and a solvate of such an ester, amide or salt: 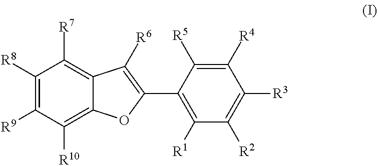
wherein R¹, R², R⁴ and R⁵ are each independently selected from the group consisting of hydrogen, OR^{A}, halogen, cyano, nitro, C₁₋₆alkyl, C₂₋₆alkenyl, C₂₋₆alkynyl, haloC₁₋₆alkyl, dihaloC₁₋₆alkyl and trihaloC₁₋₆alkyl;
R³ is selected from the group consisting of OR^{A}; —CHO; —C(O)C₁₋₄alkyl; —C(O)phenyl; —O—C(O)R^{A}; and N(R^{B})₂ in which each R^{B} is independently selected from the group consisting of hydrogen, —C(O)C₁₋₄alkyl, —C(O)phenyl, —SO₂C₁₋₄alkyl, —SO₂phenyl, C₁₋₆alkyl, C₂₋₆alkenyl, C₂₋₆alkynyl, C₃₋₈cycloalkyl, C₃₋₈cycloalkyl-C₁₋₆alkyl, C₆₋₁₀aryl, C₆₋₁₀aryl-C₁₋₆alkyl, C₅₋₁₀heterocyclyl and C₅₋₁₀heterocyclyl-C₁₋₆alkyl;
or R³ and R⁴ together with the atoms to which they are attached, form a 5-, 6- or 7-membered cyclic group optionally containing one to three heteroatoms selected from O, N and S, said 5-, 6- or 7-membered cyclic group being optionally substituted with 1 or 2 groups selected from OR^{A}, halogen, cyano, nitro, C₁₋₆alkyl, C₂₋₆alkenyl, C₂₋₆alkynyl, haloC₁₋₆alkyl, dihaloC₁₋₆alkyl and trihaloC₁₋₆alkyl;
R⁶ is selected from the group consisting of C₃₋₈cycloalkyl, C₃₋₈cycloalkyl-C₁₋₆alkyl, C₃₋₈cycloalkyl-C₁₋₆alkenyl, phenyl, biphenyl, phenyl-C(═CH₂)—, and C₅₋₁₀heterocyclyl, wherein said phenyl, biphenyl, phenyl-C(═CH₂)— or C₅₋₁₀heterocyclyl group is unsubstituted or substituted on the ring with 1 to 3 substituents, each substituent being selected from the group consisting of OR^{A}; halogen; cyano; nitro; —CHO; —CO.C₁₋₆alkyl; C₁₋₆alkyl, C₁₋₆alkoxy or C₁₋₆alkoxyalkyl optionally substituted by 1 to 3 halogen atoms; C₂₋₆alkenyl optionally substituted by halogen or cyano; C₂₋₆alkynyl; SO₂H; SO₂C₁₋₆alkyl; SH; and SC₁₋₆alkyl;
R⁸ is OR^{A};
R⁷, R⁹ and R¹⁰ are each independently selected from the group consisting of hydrogen, OR^{A}, halogen, cyano, nitro, C₁₋₆alkyl, C₂₋₆alkenyl, C₂₋₆alkynyl, C(O)H, C(O)C₁₋₆alkyl, haloC₁₋₆alkyl, dihaloC₁₋₆alkyl, trihaloC₁₋₆alkyl, cyanoC₁₋₆alkyl, and C₁₋₄alkoxyC₁₋₆alkyl; and
R^{A} is selected from the group consisting of hydrogen, C₁₋₆alkyl, C₂₋₆alkenyl, C₂₋₆alkynyl, C₃₋₈cycloalkyl, C₃₋₈cycloalkyl-C₁₋₆alkyl, C₆₋₁₀aryl and C₆₋₁₀ aryl-C₁₋₆alkyl; together with a pharmaceutically acceptable carrier.

48. The pharmaceutical composition of claim 47, in which R¹, R², and R⁵ are each independently selected from the group consisting of hydrogen, OR^{A}, halogen, cyano, nitro, C₁₋₄alkyl, haloC₁₋₄alkyl, dihaloC₁₋₄alkyl and trihaloC₁₋₄alkyl.

49. The pharmaceutical composition of claim 48, in which R¹, R² and R⁵ are each independently selected from the group consisting of hydrogen, hydroxy, halogen, cyano, methyl and trifluoromethyl.

50. The pharmaceutical composition of claim 47, in which R³ is selected from the group consisting of OR^{A} and N(R^{B})₂, in which each R^{B} is independently selected from the group consisting of hydrogen, C₁₋₄alkyl, and C₃₋₆cycloalkyl.

51. The pharmaceutical composition of claim 50, in which R³ represents a hydroxy group.

52. The pharmaceutical composition of claim 47, in which R⁴ represents hydrogen, OR^{A}, halogen, cyano, nitro, C₁₋₄alkyl, haloC₁₋₄alkyl, dihaloC₁₋₄alkyl or trihaloC₁₋₄alkyl.

53. The pharmaceutical composition of claim 52, in which R⁴ represents hydrogen, hydroxy, halogen, cyano, methyl or trifluoromethyl.

54. The pharmaceutical composition of claim 47, in which R³ and R⁴ together represent an —NH—CH═N—, —NH—N═CH— or —CH—CH—NH— group.

55. The pharmaceutical composition of claim 47, in which R⁶ is selected from the group consisting of C₃₋₆cycloalkyl, C₃₋₆cycloalkyl-C₁₋₂alkyl, C₃₋₆cycloalkyl-C₁₋₂alkenyl, phenyl, biphenyl, phenyl-C(═CH₂)—, and C₅₋₇heterocyclyl wherein said phenyl, biphenyl, phenyl-C(═CH₂)— or C₅₋₁₀heterocyclyl group is unsubstituted or substituted on the ring with 1 or 2 substituents, each substituent being selected from the group consisting of OR^{A}; halogen; cyano; and C₁₋₄alkyl or C₁₋₄alkoxy optionally substituted by 1 to 3 halogen atoms.

56. The pharmaceutical composition of claim 55, in which R⁶ is an aromatic group.

57. The pharmaceutical composition of claim 55, in which R⁶ is an optionally substituted phenyl or C₅heterocyclyl group.

58. The pharmaceutical composition of claim 47, in which R⁷, R⁹ and R¹⁰ are each independently selected from the group consisting of hydrogen, OR^{A}, halogen, cyano, C₁₋₄alkyl, haloC₁₋₄alkyl, dihaloC₁₋₄alkyl and trihaloC₁₋₄alkyl.

59. The pharmaceutical composition of claim 58, in which R⁷, R⁹ and R¹⁰ are each independently selected from hydrogen, hydroxy, halogen, cyano, methyl and trifluoromethyl.

60. The pharmaceutical composition of claim 47, in which R^{A} is selected from the group consisting of hydrogen, C₁₋₄alkyl, C₃₋₆cycloalkyl, C₃₋₆cycloalkyl-C₁₋₄ alkyl, phenyl and benzyl.

61. The pharmaceutical composition of claim 60, in which R^{A} represents hydrogen or C₁₋₄alkyl.

62. The pharmaceutical composition of claim 47, in which:
R^{A} represents C₁₋₄alkyl or hydrogen;
R¹, R², and R⁵ are each independently selected from the group consisting of hydrogen, OR^{A}, halogen, cyano, halomethyl, dihalomethyl and trihalomethyl;
R³ represents OR^{A} or N(R^{B})₂, in which each R^{B} independently represents hydrogen or C₁₋₄alkyl;
R⁴ represents one of the preferred groups mentioned above for R¹, R², and R⁵; or
R³ and R⁴ together represent an —NH—N═CH— group;
R⁶ represents phenyl or a C₅heterocyclyl group which can either be unsubstituted or substituted on the ring with 1 or 2 substituents, each substituent being selected from the group consisting of OR^{A}; halogen; cyano; and C₁₋₄alkyl or C₁₋₄alkoxy optionally substituted by 1 to 3 halogen atoms; and
R⁷, R⁹ and R¹⁰ each independently represent hydrogen, OR^{A}, halogen, cyano, halomethyl, dihalomethyl and trihalomethyl.

63. The pharmaceutical composition of claim 62, in which at least one of R¹, R², and R⁵ represents hydrogen; R³ represents OR^{A}; or R³ and R⁴ together represent an —NH—CH═N—, —NH—N═CH— or —CH═CH—NH— group; R⁶ represents phenyl or a C₅heterocyclyl group optionally substituted by cyano; and R⁷, R⁹ and R¹⁰ each independently represent hydrogen, hydroxy, halogen, cyano, methyl and trifluoromethyl, at least one of R⁷, R⁹ and R¹⁰ being hydrogen.

64. A pharmaceutical composition comprising a compound as claimed in claim 44, together with a pharmaceutically acceptable carrier.

65. A method for the treatment or prophylaxis of a disease or disorder associated with estrogen receptor activity in a mammal, which comprises administering to the mammal a therapeutically effective amount of the composition of claim 47.

66. The method of claim 65, wherein the condition associated with a disease or disorder associated with estrogen receptor activity is selected from bone loss, bone fractures, osteoporosis, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression, autoimmune disease, inflammation, IBD, IBS, sexual dysfunction, hypertension, retinal degeneration and lung, colon, breast, uterus, and prostate cancer.
